(19) United States
(12) Patent Application Publication
(10) Pub. No.: US 2024/0299747 A1
(43) Pub. Date: Sep. 12, 2024

(54) ACUTE BLOCKADE WITH DELAYED NEURAL SUPPRESSION

(71) Applicant: Presidio Medical, Inc., South San Francisco, CA (US)

(72) Inventors: Douglas Michael Ackermann, Reno, NV (US); Michael A. Faltys, San Francisco, CA (US); James Harris, San Francisco, CA (US); Thomas Flores, San Francisco, CA (US); Kenneth S. Wu, San Francisco, CA (US)

(21) Appl. No.: 18/563,859

(22) PCT Filed: May 26, 2022
(86) PCT No.: PCT/US2022/031163

§ 371 Date: Nov. 22, 2023

Related U.S. Application Data
(60) Provisional application No. 63/193,541, filed on May 26, 2021.

(51) Int. Cl. A61N 1/36; A61N 1/04; A61N 1/05; A61N 1/20

(52) U.S. Cl. CPC A61N 1/36071; A61N 1/0456; A61N 1/0492; A61N 1/0551; A61N 1/20; A61N 1/36153; A61N 1/36164

(57) ABSTRACT

Disclosed herein are systems and methods for electrically modulating tissue. Systems can include a current generator; at least one implantable working electrode, the at least one implantable working electrode configured to be in electrical communication with the current generator; at least one indifferent electrode; and a controller configured to signal the current generator to: generate a set of currents with a set of initial polarities to be delivered to the working electrodes; and wherein the at least one indifferent electrode absorbs a bias current which is equal in magnitude and opposite in polarity to a summation of the set of currents.

### BACKGROUND

This application relates, in some embodiments, to facilitating block, modulation or attenuation of biological signals through nerve tissue, including the processing of biological tissue in nervous system tissue, cardiac tissue, or other voltage-sensitive tissue.

The gate control theory of pain was developed in the 1960s and led to the advent of stimulation-based pain management therapies to reduce pain inputs from reaching the brain by selectively stimulating non-nociceptive fibers (non-pain transmitting fibers) in the spinal cord to inhibit transmission of pain stimuli to the brain (See Mendell, Constructing and Deconstructing the Gate Theory of Pain, Pain, 2014 February 155(2): 210-216). Current stimulation systems for spinal cord stimulation (SCS), which act on this gate control theory to indirectly reduce pain, typically have relied on stimulation signals in the <100 Hz frequency range, and recently in the kHz frequency range. Stimulation of the dorsal root ganglia, DRG, in a similar frequency range has also been employed to reduce segmental pain through the same mechanism.

However, technologies based on this premise have drawbacks as pain transmission inhibition is not complete and side effects such as paresthesia can be uncomfortable for patients. Therefore, it is desirable to have systems and methods of treating pain (or otherwise blocking neural activity) which more effectively block or attenuate pain signal transmission through pain fibers, or decrease the excitability of neurons which process pain signals, rather than indirectly reducing pain signals through gate-theory activation of non-nociceptive fibers, as well as avoid undesirable side effects. Furthermore, block or attenuation of neural tissue or neural activity has been implicated in not only affecting pain but also in the management of movement disorders, psychiatric disorders, cardiovascular health, as well as management of disease states such as diabetes.

### SUMMARY

In one embodiment, a method or system for acute blockade with delayed neural suppression is provided. In one embodiment, a system for electrically modulating tissue includes: a current generator; at least one implantable working electrode, the at least one implantable working electrode configured to be in electrical communication with the current generator; at least one indifferent electrode; and a controller configured to signal the current generator to: generate a set of currents with a set of initial polarities to be delivered to the working electrodes for a time period; and wherein the magnitudes of the currents are less than a threshold current amplitude corresponding to a current amplitude required to achieve acute blockade in neural tissue, and cause neural suppression in the neural tissue after the time period. In one embodiment, a method for electrically modulating tissue includes: providing a current generator; signaling the current generator (e.g., with a controller or otherwise) to generate a set of currents with a set of initial polarities to be delivered to the working electrodes for a time period, wherein the magnitudes of the currents are less than a threshold current amplitude corresponding to a current amplitude required to achieve acute blockade in neural tissue; and causing neural suppression in the neural tissue after the time period.

The time period may be a predetermined time period. The controller may be configured to signal the current generator to generate the set of currents in a closed loop mode of operation, an open loop mode of operation, or other mode of operation. The controller may be configured to signal the current generator to cause neural suppression in the neural tissue after the time period and without causing sensory deficit. The set of currents may be sufficient to modulate electrically excitable tissue.

The at least one implantable working electrode may include a high charge capacity material. The controller may be further configured to signal the current generator to generate a bias current. The bias current may operate in an anodic polarity or in a cathodic polarity. The current generator may be configured to generate the bias current that biases the working electrode voltages cathodically. The current generator may be configured to generate the bias current that biases the indifferent electrode voltages anodically. The system may include at least two working electrodes. An initial polarity of at least one working electrode may be cathodic or anodic.

The indifferent electrode may be a skin surface electrode. The indifferent electrode may be a transcutaneous electrode or an implanted electrode. The indifferent electrode may include titanium. The indifferent electrode may have a working surface area of at least about 10 cm², or at least about 50 cm². The system may be configured to generate a block of the excitable tissue. The electrode may include titanium nitride, tantalum, MP35N, platinum iridium, stainless steel, and/or a combination thereof. The electrode may include platinum-iridium coated with titanium nitride.

The system may not include any blocking capacitors. The bias current may be fixed or variable. The bias current may be variable over a cycle period or from a first cycle to a second cycle. The controller may be configured to adjust the set of working electrode currents to modulate the bias current based on receiving data relating to one or any combination of capacitance, maximum current, and voltage. The bias current may be between about −10 μA and about −1 mA, or between −10 μA and about −100 μA.

The bias current may be evenly or unevenly split between a plurality of the implantable working electrodes. The system may be further configured to inhibit water electrolysis in the patient. The system may be further configured to inhibit one or more of: corrosion of the working electrode, an undesired chemical reaction, hydrogen embrittlement, creation of a reactive oxygen species, or creation of hydrogen peroxide.

The current generator may be configured to generate DC current and/or high frequency AC current. The working electrode may be configured to deliver at least about 2,000 μC of charge into excitable tissue without damaging the excitable tissue. The system may be devoid of any mechanically moving parts. The time period may be selected from the group consisting of: at least 5 minutes, at least 10 minutes, at least 20 minutes, at least 30 minutes, at least 45 minutes, at least one hour, at least two hours, at least 3 hours, at least four hours, and at least 5 hours. The threshold current amplitude between 700 and 1000 μA. The neural suppression may include pain suppression. The sensory deficit may be selected from the group consisting of: numbness, anesthesia, and paresthesia.

In another embodiment, a method or system for reduced pain response by reducing windup response while maintaining acute pain response is provided. A system for electrically modulating tissue may include: a current generator; at least one implantable working electrode, the at least one implantable working electrode configured to be in electrical communication with the current generator; at least one indifferent electrode; and a controller configured to signal the current generator to: generate a set of currents with a set of initial polarities to be delivered to the working electrodes for a time period; cause a reduction in hypersensitive neuron sensitivity; and maintain an acute pain response. A method for electrically modulating tissue may include: providing a current generator; signaling the current generator (e.g., with a controller or otherwise) to generate a set of currents with a set of initial polarities to be delivered to the working electrodes for a time period; causing a reduction in hypersensitive neuron sensitivity; and maintaining an acute pain response.

The time period may be a predetermined time period. The controller may be configured to signal the current generator to generate the set of currents in a closed loop mode of operation, an open loop mode of operation, or other mode of operation. The set of currents may be sufficient to modulate electrically excitable tissue. The at least one implantable working electrode may include a high charge capacity material. The bias current may operate in an anodic or cathodic polarity. The set of currents may be configured to generate the bias current that biases the working electrode voltages cathodically or anodically. The set of currents may be configured to generate the bias current that biases the indifferent electrode voltages cathodically or anodically.

The system may include at least two working electrodes. The initial polarity of at least one working electrode may be cathodic or anodic. The indifferent electrode may be a skin surface electrode. The indifferent electrode may be a transcutaneous electrode or an implanted electrode. The indifferent electrode may include titanium. The indifferent electrode may have a working surface area of at least about 10 cm² or at least about 100 cm².

The system may be configured to generate a block of the excitable tissue. The at least one implantable working electrode may include titanium nitride, tantalum, MP35N, and/or a combination thereof. The at least one implantable working electrode may include tantalum coated with titanium nitride. The system may not include any blocking capacitors. The bias current may be fixed or variable. The controller may be configured to adjust the set of working electrode currents to modulate the bias current based on receiving data relating to one or any combination of capacitance, maximum current, and voltage. The bias current may be between about −10 μA and about −1 mA or between about −10 μA and about −100 μA. The bias current may be evenly or unevenly split between a plurality of the implantable working electrodes.

The system may be configured to inhibit water electrolysis in the patient. The system may be configured to inhibit corrosion of the working electrode. The current generator may be configured to generate DC current. The current generator may be configured to generate high frequency AC current. The working electrode may be configured to deliver at least about 2,000 μC of charge into excitable tissue without damaging the excitable tissue. The system may be devoid of any mechanically moving parts. The time period may be selected from the group consisting of: at least 5 minutes, at least 10 minutes, at least 20 minutes, at least 30 minutes, at least 45 minutes, at least one hour, at least two hours, at least 3 hours, at least four hours, and at least 5 hours.

In another embodiment, a method or system for controlling current amplitude to achieve partial block is provided. A system for electrically modulating tissue may include: a current generator; at least one implantable working electrode, the at least one implantable working electrode configured to be in electrical communication with the current generator; at least one indifferent electrode; and a controller configured to signal the current generator to: generate a set of currents with a set of initial polarities and current amplitude levels associated with a partial neural conduction block of neural tissue to be delivered to the working electrodes; and cause a partial neural conduction block in the neural tissue in response to said set of currents. A method for electrically modulating tissue may include: providing a current generator; signaling the current generator (e.g., with a controller or otherwise) to generate a set of currents with a set of initial polarities and current amplitude levels associated with a partial neural conduction block of neural tissue to be delivered to the working electrodes; and causing a partial neural conduction block in the neural tissue in response to said set of currents.

The set of currents may be sufficient to modulate electrically excitable tissue. The at least one implantable working electrode may include a high charge capacity material. The bias current may operate in an anodic or cathodic polarity. The set of currents may be configured to generate the bias current that biases the working electrode voltages cathodically or anodically. The set of currents may be configured to generate the bias current that biases the indifferent electrode voltages cathodically or anodically.

The may include at least two working electrodes. The initial polarity of at least one working electrode may be cathodic or anodic. The indifferent electrode may be a skin surface electrode, a transcutaneous electrode or an implanted electrode. The indifferent electrode may include titanium. The indifferent electrode may have a working surface area of at least about 10 cm² or at least about 100 cm².

The system may be configured to generate a block of the excitable tissue. The at least one implantable working electrode may include titanium nitride, tantalum, MP35N, and/or a combination thereof. The at least one implantable working electrode may include tantalum coated with titanium nitride. The system may not include any blocking capacitors.

The bias current may be fixed or variable. The controller may be configured to adjust the set of working electrode currents to modulate the bias current based on receiving data relating to one or any combination of capacitance, maximum current, and voltage. The bias current may be between about −10 μA and about −1 mA, or between about −10 μA and about −100 μA. The bias current may be evenly or unevenly split between a plurality of the implantable working electrodes.

The system may be configured to inhibit water electrolysis in the patient. The system may be configured to inhibit corrosion of the working electrode. The current generator may be configured to generate DC current or high frequency AC current. The working electrode may be configured to deliver at least about 2,000 μC of charge into excitable tissue without damaging the excitable tissue. The system may be devoid of any mechanically moving parts.

The set of currents may include a positive plateau phase and a negative plateau phase, and wherein the current amplitude level is selected to cause a neural block during only the positive plateau phase. The set of currents may include a positive plateau phase and a negative plateau phase, and wherein the current amplitude level is selected to cause a neural block during only the negative plateau phase. The set of currents may include a positive plateau phase and a negative plateau phase, and wherein the current amplitude level is selected to cause a neural block during both the positive plateau phase and the negative plateau phase. The set of currents may be chosen to cause a neural block during both the positive plateau phase and the negative plateau phase, while allowing neural conduction during the transition period between phases.

The current amplitude level may be linearly related to a percentage of partial block. The current amplitude level may be exponentially related to a percentage of partial block. The controller may be further configured to determine an indication of a level of neural blocking and adjust the amplitudes of the set of currents in response to the indication. The controller may be further configured to cause the partial neural conduction block to be eliminated within 10, 20, 30, 40, 50, 60, 70, 80, or 90 ms of stopping generation of the set of currents. The controller may be configured to cause the partial neural conduction block to be eliminated within a period of time proportional to the period of time that the partial block was applied. For example, the controller may be configured to cause the partial neural conduction block to be eliminated within 50%, 60%, 70%, 80%, 90%, 100%, 110%, 120%, 130%, 140%, 150%, or 200% of a period of time that the partial neural conduction block is applied.

A system for electrically modulating tissue may include any one or more of the embodiments described in the disclosure. A method for electrically modulating tissue may include any one or more of the embodiments described in the disclosure.

### BRIEF DESCRIPTION OF THE DRAWINGS

FIG. 1 schematically illustrates an embodiment of a waveform between working electrodes with a constant bias current.

FIG. 2 schematically illustrates one non-limiting example of a neuromodulation system.

FIG. 3 schematically illustrates an embodiment of the percutaneous lead of the neuromodulation system of FIG. 2.

FIG. 4 schematically illustrates an embodiment of the lead adaptor of the neuromodulation system of FIG. 2.

FIG. 5 schematically illustrates an embodiment of the external pulse generator (EPG) of the neuromodulation system of FIG. 2.

FIG. 6 schematically illustrates an example of an EPG Output Waveform that may be provided by the neuromodulation system of FIG. 2.

FIG. 7 schematically illustrates a surface electrode carriage device.

FIG. 8 schematically illustrates one embodiment of a sterile lead implantation kit.

FIG. 9A illustrates spinal cord stimulation (SCS) waveforms.

FIG. 9B illustrates an SCS waveform.

FIG. 9C illustrates a table of values related to various SCS waveforms.

FIG. 10 illustrates an example waveform.

FIG. 11 illustrates an example waveform of an electrode operated in ranges to limit chemical by products and protect the electrode from undesirable reactions.

FIG. 12 illustrates an example bipolar operation with bias (or offset) current.

FIG. 13 illustrates a recording from a filament with ectopic firing in a neuropathic state.

FIG. 14 illustrates a raster plot showing the block and recovery of conduction in the filament referred to in reference to FIG. 12.

FIG. 15 illustrates a distribution plot showing block and recovery times in test on sample fibers where full conduction block was achieved.

FIG. 16 illustrates recording from a filament containing two A fibers activated by electrical stimulation (foot shock-evoked).

FIG. 17 illustrates a cross-section of a spinal cord and lead.

FIG. 18 illustrates ULF current and neurogram results with a slowly developing block in a filament containing 4 A fibers that are activated by electrical stimulation (foot shock-evoked).

FIG. 19 illustrates a histogram showing the percentages of different fiber types in which conduction could be blocked at currents up to 2000 μA or others.

FIG. 20 illustrates thresholds for block and recovery from current application in different fiber types.

FIG. 21 illustrates a recording of the block of foot shock-evoked responses in a dorsal horn neuron by DC applied to a cord surface.

FIG. 22 is a schematic representation of the node of Ranvier with illustrated ion channels and paths.

FIG. 23 illustrates a raster sweep for a computer simulation illustrated in FIG. 22.

FIG. 24 illustrates a membrane voltage (mV) over time in seconds showing gradual decrease in AP conduction with ULF in a computer simulation.

### DETAILED DESCRIPTION

This application describes, in some aspects, methods and systems for management of chronic and acute pain states via application of direct current (DC) to facilitate nerve block or attenuation including nerve hypersuppression, or nerve block without rapid reversibility or recovery after direct current application has been removed or stopped. What is disclosed in some embodiments are systems and electrodes for delivering blocking or attenuating direct current (DC) to neural tissue by delivering cycled cathodic and anodic current. In some embodiments these systems involve high-charge capacity materials. Tissue safety can be maintained by operating the electrode below reaction potentials for undesired reactions as well as by limiting the amount of irreversible reactions, such as electrolysis of water, or oxidation and reduction of water (H₂O), which create harmful reactive species such as OH−, H+ or oxygen free radicals, and in some cases by maintaining a bias current, as discussed further herein. More generally, neuromodulation as described within refers to modulation of activity, excitability or cellular state of neural tissue (including but not limited to neurons and/or glial cells), cardiac tissue or other neural or non-neural excitable tissue or tissue which can be impacted by electrical fields or currents. In some embodiments, either the anodic or cathodic phases of a delivered waveform to a patient, or both the anodic and cathodic phases can have a therapeutic effect on electrically excitable tissue, such as neural tissue for example.

Not to be limited by theory, the propagation of action potentials in electrically excitable tissue, e.g., neural tissue, leads to refractory periods on the order of milliseconds for sodium channels, typically between about 1 ms and about 20 ms, or between about 2 ms and about 5 ms for the combined absolute and relative refractory periods, thus (e.g., ultra low frequency) AC current waveforms with half periods meaningfully greater than this refractory period (e.g., greater than about 1 ms, 1.5 ms, 2 ms, 2.5 ms, 3 ms, 10 ms, 30 ms, 50 ms, 100 ms, 300 ms, 500 ms, 1000 ms, 2000 ms, 5000 ms, 6000 ms or more) and having sufficiently low differential rates (e.g., rise and fall-times) to not induce action potentials can also be used to create tissue blockade or attenuation, and will be perceived by electrically excitable tissue as a direct current stimulus. As such, direct current (DC) as defined herein is inclusive of ultra low frequency AC current waveforms that are perceived as and functionally are direct current from the perspective of the tissue whose action potentials or neural processing is being modulated. The ultra low frequency could be, for example, less than about 10 Hz, 9 Hz, 8 Hz, 7 Hz, 6 Hz, 5 Hz, 4 Hz, 3 Hz, 2 Hz, 1 Hz, 0.5 Hz, 0.1 Hz, 0.05 Hz, 0.01 Hz, 0.005 Hz, 0.0001 Hz, or ranges including any two of the foregoing values so long as the direction of current flow is constant over at least the entire refractory period of the target tissue, or at least twice as long, or at least five times as long, or at least ten times as long as the refractory-causing membrane channel time constant (for example, fast sodium channel inactivation gate time constant).

Chronic pain is a significant burden on individuals and society as a whole. Nearly 50 million adults are estimated to have significant chronic or severe pain in the U.S. alone. (See Nahin, Estimates of Pain Prevalence and Severity in Adults: United States, 2012, The Journal of Pain, 2015 Aug. 16(8): 769-780). Worldwide, chronic pain is estimated to affect more than 1.5 billion people. (Borsook, A Future Without Chronic Pain: Neuroscience and Clinical Research, Cerebrum, 2012 June). While surgical techniques are sometimes applied to remove a specific source of pain, frequently due to impingement of a nerve, in many cases the precise cause of pain is not clear and cannot be reliably addressed via a surgical procedure. Pain management can alternatively be addressed by overwhelming the central nervous system with stimulating signals that prevent registration of pain inputs (gate control theory of pain). Typically, this stimulation, such as in the case of spinal cord stimulation (SCS), is performed using metal electrodes and alternating current (AC) stimulation to produce these additional stimulating signals to prevent pain sensation. However, one major drawback is the presence of paresthesia, a sensation of tingling in the innervated region downstream from the stimulated nerve. Methods to eliminate paresthesia, which patients can find discomforting, have led to different means of stimulation from conventional tonic SCS (˜30-120 Hz) stimulation including high frequency stimulation (˜10 kHz) and burst stimulation (e.g., five pulses at 500 Hz delivered 40 times per second). (Tjepkema-Cloostermans et al, Effect of Burst Evaluated in Patients Familiar With Spinal Cord Stimulation, Neuromodulation, 2016 Jul. 19(5):492-497).

An alternative means to manage pain signaling to the central nervous system is to prevent conduction of the pain signals from the peripheral signal source by directly blocking or attenuating the pain signals as compared to masking the pain signals by generating alternative neural inputs to crowd out and inhibit pain signal transmission as in traditional SCS and gate theory. One means to do this is by applying a direct current (DC) to a nerve to prevent action potential (AP) generation and transmission. Because this does not stimulate the nerve as in traditional stimulation, paresthesia can be avoided. The mechanism leading to AP block has been attributed to a depolarization block or hyperpolarization block that deactivates the sodium channels required for an action potential event under the electrode site. (See Bhadra and Kilgore, Direct Current Electrical Conduction Block of Peripheral Nerve, IEEE Transactions on Neural Systems and Rehabilitation Engineering, 2004 Sep. 12(3): 313-324). Wide dynamic range (WDR) neurons integrate pain signals and have also been implicated as a contributing source of pain in patients, and application of direct current (DC) is well positioned to reduce this activity and may impact associated inhibitory and excitatory neurons that drive WDR activity.

The unmitigated use of direct current has long been known to be dangerous to nerve tissue due to creation of toxic species at the electrode-nerve interface. As such, systems and methods that facilitate safe delivery of direct current therapy would be highly desirable. In some embodiments, systems and methods can be configured to treat nociceptive pain. In some embodiments, systems and methods of treating pain and other medical can involve selective blockade of antero-lateral column tissue in the spinal cord. Furthermore, some embodiments relate to systems and methods of treating pain by the aforementioned systems and methods, specifically through selective blockade of dorsal root tissue and/or dorsal root ganglia. Moreover, in some embodiments, disclosed herein are systems and methods of treating pain, specifically through blockade or attenuation of one or more peripheral nerves.

In some embodiments, systems and methods can safely block or attenuate pain signals (which includes modulation of pain processing) in the spinal column by delivering very low frequency (e.g., ultra low frequency) stimulation in the epidural space for up to two weeks or more, to achieve clinically measurable pain reduction in patients with chronic low back pain who are candidates for spinal cord stimulation (SCS).

With targeted nerve block, pain from specific dermatomes and pain in regional body sites can be managed. A number of localized targets implicated in moderating pain signal transduction can be addressed. For example, both more centrally located nerve tissues such as the spinothalamic tract and dorsal root ganglion can be targeted to manage lower back pain, sciatica, and complex regional pain syndrome (CPRS) among other pain considerations.

In some embodiments, an electrode (e.g., working electrode, indifferent electrode, or any other electrode described herein) can include a contact comprising a high charge-capacity material and/or any other material described herein. The electrode contact can have in some cases a geometric surface area of between about 1 mm² and about 10 mm², or about 1 mm². 2 mm², 3 mm², 4 mm², 5 mm², 6 mm², 7 mm², 8 mm², 9 mm², 10 mm², 20 mm², 50 mm², 100 mm², or ranges including any two of the foregoing values. The electrode contact itself can be fabricated of a high charge capacity material, such as those described, for example, in U.S. Pat. No. 10,071,241 to Bhadra et al., which is hereby incorporated by reference in its entirety. Alternatively, the electrode contact can comprise a base at least partially, or entirely coated with a high charge capacity material. In some embodiments, a high charge capacity material can have a Q value of at least about 25, 50, 100, 200, 300, 400, 500, 1,000, 2,500, 5,000, 10,000, 50,000, 100,000, 500,000, or more μC, or ranges including any two of the foregoing values. The Q value of an electrode contact can refer to the total amount of charge that can be delivered through an electrode contact before the electrode contact begins generating irreversible chemical reactions at a rate that cannot be cleared through the body's nominal transport mechanism. These chemical reactions include but are not limited to oxygen or hydrogen evolution, or dissolution of the electrode materials. Non-limiting examples of high charge capacity materials are titanium grade 2, platinum black, iridium oxide, titanium nitride, tantalum, silver chloride, poly(ethylenedioxythiophene) and suitable combinations thereof. In some embodiments, one or more electrodes include fluorine-free tantalum carbide MXene (two-dimensional transition metal carbide) hybrid structures. The electrodes can comprise fractal coatings or high surface area formats in some embodiments. High charge capacity materials may be configured to be monolithic or as coatings on base substrates. Non-limiting examples of substrates for coating include stainless steel such as 304 and 316LVM, nickel-colbalt-chrome alloys such as MP35N®, platinum and platinum-iridium, platinum-iridium (90/10), platinum-iridium (80/20), titanium, nickel-titanium alloys such as Nitinol. In some embodiments, the electrodes can include tantalum coated with titanium nitride. Tantalum as one non-limiting example can be a particularly advantageous material for its superior radiopacity, thus allowing for improved implantation, verification, and/or removal of implantable neuromodulation devices. To generate more surface area for the electrochemical reactions to occur, the traditional electrodes may be made from high surface area to volume structures such as electroplated surfaces, (e.g., platinum black/electrodeposited iridium oxide for example), roughened surfaces, woven surfaces, patterned surfaces, reticulated foam structures, porous sintered bead structures, nano- or micro-patterned structures to expose additional material surface area. In some embodiments, the electrode can be a SINE (separated-interface nerve electrode) or EICCC (electron to ion current conversion cell) electrode in which an electrode is immersed in an electrolyte solution which is in contact with an ion-conductive material-electrolyte solution interface with an ion-conductive material that electrically contacts the cardiac tissue or area proximal to cardiac tissue, as described, for example, in U.S. Pat. No. 9,008,800 to Ackermann et al., and U.S. Pub. No. 2018/0280691 to Ackermann et al., which are hereby incorporated by reference in their entireties.

In some embodiments, disclosed herein are systems and methods for safely and efficaciously stimulating neural tissue that can advantageously utilize a variety of waveforms from DC to high frequencies. Stimulation with DC, although potentially very useful, has not been commercially utilized for neural modulation because neurostimulation systems capable of delivering DC safely for long periods of time have not been available. Available commercial systems prevent DC delivery to limit irreversible electrochemical reactions, relying on charge balancing mechanisms. These systems can include blocking the DC component with capacitors, blocking capacitors (coupling capacitors), or mechanisms that remove charge accumulation at the end of a stimulation cycle. While reliable, typical capacitors limit charge to less than about one millicoulomb (mC) per phase, disallowing the use of ultra-low frequency signals at large charge magnitudes in excess of this charge capacity. The other widely utilized technique relies on actively balanced current sources, but these require redundancy to be fault tolerant and typically do not deliberately control electrode voltages important for some electrode technologies and have not been shown to be advantageous for long-term high charge delivery. Active systems in conjunction with coatings have been utilized in such devices as retinal implants to increase charge densities to about ˜2 mC/cm², but these densities are still insufficient to allow use of very high charge per phase waveforms required by DC, ultra low frequency, or very low frequency waveforms with sufficient current amplitude.

Some embodiments involve high surface area electrode coatings in conjunction with a bias current such as, for example, a DC bias to maintain the electrode voltages in the optimal range for a particular electrode material for long term operational durability. This approach can boost the charge per phase from about 50 μC/cm² used in conventional systems to about or at least about 5,000 μC/cm², 25,000 μC/cm², 50,000 μC/cm², and beyond in some cases without, for example, causing damage to either the electrode or the electrically excitable tissue. Systems and methods configured to allow for an intentional net bias current, e.g., DC bias, such as via a control system, can, in some cases, advantageously maintain the health of the high charge capacity electrodes (by preventing or inhibiting corrosion, e.g., oxidation, or other damage to the electrodes) as well as minimizing or preventing undesired reactions and generation of species such as OH−, H+ or oxygen free radicals that can lead to tissue damage. Peroxide formation is another non-limiting example of an undesired reaction that can be prevented or inhibited with systems and methods as disclosed herein, as well as other undesired reactions depending on the material (e.g., metallic material or alloy, for example) and its location.

In some embodiments, the charge per anodic and/or cathodic phase is, for example, about 1,000 μC, 1,500 μC, 2,000 μC, 2,500 μC, 3,000 μC, 3,500 μC, 4,000 μC, 4,500 μC. 5,000 μC, 5,500 μC, 6,000 μC or more or less, such as between about 4,000 μC and about 5,000 μC per phase, and ranges including any two of the foregoing values.

In some embodiments, systems and methods for the delivery of current via implanted electrodes do not include capacitors such as blocking capacitors (coupling capacitors). In some embodiments, systems and methods for the delivery of current via implanted electrodes do not include resistors.

In some embodiments, the bias current is the current resulting from the summation of the currents being simultaneously delivered to the electrode contacts or working electrodes in proximity to the target excitable or voltage-sensitive tissue. In some embodiments, the bias current is equal in magnitude and opposite in polarity to the summation of the currents being simultaneously delivered to the electrode contacts or working electrodes. In some embodiments, the currents being simultaneously delivered to the electrode contacts or working electrodes can be adjusted to modulate the bias current.

In some embodiments, the bias current can be a cathodic bias current (for example, for TiN (titanium nitride) or tantalum electrodes). In some embodiments, the bias current can be an anodic bias current, with (+) positive values substituted for any (−) values disclosed herein (for example, for IrOx electrodes). In some embodiments, the total bias current can be, for example, between about −10 μA and about −1 mA, between about −10 μA and about −100 μA, between about −0.01 μA and about −1000 μA, between about −0.01 μA and about −0.1 μA, between about −0.1 μA and about −1 μA, between about −1 μA and about −1000 μA, between about −20 μA and about −100 μA, or about −0.01 μA, −0.05 μA, −0.10 μA, −0.50 μA, −1 μA, −5 μA, −10 μA, −15 μA, −20 μA, −25 μA, −30 μA, −35 μA, −40 μA, −45 μA, −50 μA, −55 μA, −60 μA, −65 μA, −70 μA, −75 μA, −80 μA, −85 μA, −90 μA, 95 μA, −100 μA, or more or less, or ranges including any two of the foregoing values. The bias current can be, for example, values as disclosed herein, and in some embodiments between about −40 μA and about −50 μA, such as about −40 μA, −41 μA, −42 μA, −43 μA, −44 μA, −45 μA, −46 μA, −47 μA, −48 μA, −49 μA, −50 μA, and ranges including any two of the foregoing values. As noted, the bias current can be either negative as noted in some values and ranges herein, or alternatively positive (e.g., the absolute value) of any of the values or ranges of values disclosed herein, including but not limited to those in this paragraph. In some embodiments, the total bias current can be split, e.g., evenly among the number N of working electrodes. For example, in a bipolar system with 2 working electrodes, the bias current of each working electrode can be the total bias current/2, such as −50 μA/2=−25 μA or +50 μA/2=+25 μA in some embodiments, where the total bias current is the offset from the perspective of the indifferent electrode, and the bias current/N is from the perspective of the working electrode, wherein N is the number of working electrodes. The bias current may be even larger or smaller in amplitude, e.g., to accommodate use in embodiments with larger or smaller electrode surface areas.

In some embodiments, the bias current can be fixed. In some embodiments, the bias current can be variable, and be altered depending on voltage and/or other measurements (e.g., when a voltage waveform exhibits linearity or non-linearity, for example).

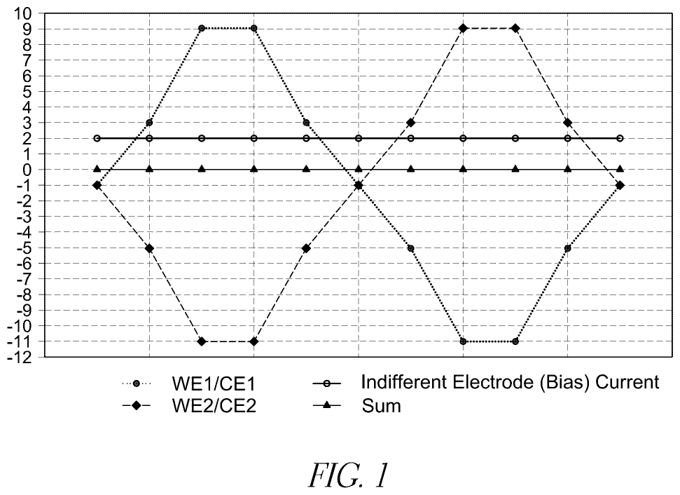

FIG. 1A schematically illustrates an embodiment of a waveform between working electrodes with a constant bias current. As illustrated, the example waveform is bipolar, and each biased down by 1 unit of current leading to a total bias current of 2 units, and all currents sum to zero. Other bias magnitudes can be as described, for example, elsewhere herein.

In some embodiments, the bias current resulting from the working electrode currents can be delivered to one, two, or more indifferent electrodes (e.g., only a single indifferent electrode in some embodiments). The indifferent electrode may be configured to be a passive current sink or source or alternatively may be an active current sink or source. In some embodiments, the indifferent electrode(s) can have a relatively high working surface area to minimize the current density on the electrode surface and minimize or prevent discomfort or other sensations of the delivered current to the patient. The indifferent electrode may be implanted, be a transcutaneous electrode, or be a body surface electrode. In some embodiments, the indifferent electrode can have a working surface area of about or at least about 1 cm², 5 cm², 10 cm², 25 cm², 50 cm², 75 cm², 100 cm², 125 cm², 150 cm², 175 cm², 200 cm², 500 cm², 1000 cm², or ranges including any two of the foregoing values.

A transcutaneous indifferent electrode can be skin adherent utilizing, for example, adhesives, and be configured to pass offset/bias current continuation for the wear duration without substantially increasing voltage. The transcutaneous indifferent electrode may comprise electrically conductive materials such as hydrogels and the like in combination with adhesives to promote skin adherence. The wear duration can be, for example, at least 3, 4, 5, 6, 7, 8, 9, 10, 11, 12, 13, 14, or more days, or ranges including any two of the foregoing values, such as between about 7-10 days in some cases.

In some embodiments, a transcutaneous indifferent electrode can have a relatively low DC resistance (without skin resistance), such as, for example, less than about 1,000, 900, 800, 700, 600, 500, 400, 300, 200, 100, 90, 80, 70, 60, 50, 40, 30, 20, 10 Ohms, or more or less, and ranges including any two of the foregoing values.

In some embodiments, the increase in driving voltage of the system from the initial driving voltage will be less than 100 mV, 200 mV, 300 mV, 400 mV, 500 mV, 600 mV, 700 mV, 800 mV, 900 mV, 1,000 mV, 1,100 mV, 1,200 mV, 1,300 mV, 1,400 mV, 1,500 mV or more over the wear duration.

In some embodiments, an implantable indifferent electrode can be the housing, e.g., can of an implantable pulse generator of a neuromodulation system and may comprise titanium, e.g., grade 1, grade 2, or grade 4 titanium as non-limiting examples, or platinum or platinum plated titanium.

Due to their exponential nature as a function of voltage, irreversible electro-chemical reactions cannot be completely avoided. These reactions need to be limited such that the byproducts can be removed by the transport properties of surrounding tissues including diffusion and fluid flow.

Reversible electro-chemical reactions need to be reversed and byproducts recovered to the extent possible such that their byproducts can be removed by the surrounding tissues without accumulating to remain below tissue toxicity levels. The rate of removal is dependent upon the location in the body and the mechanical and electromechanical characteristics of the electrode.

Charge can be categorized as recoverable and un-recoverable. One important goal in some cases is to minimize the un-recoverable charge to a level that the body can safely transport away generated byproducts resulting from reactions due to the un-recoverable charge to minimize tissue damage.

Electro-chemical reactions exponentially increase with voltage potential, therefore controlling voltages on electrode surfaces can be an effective way to minimize undesired electro-chemical reactions. These voltage potentials can be controlled by biasing the electrode with DC or similar current.

Electrodes designed to produce uniform current densities (or relatively uniform) allow the use of average voltage to be representative of the electro-chemical reactions.

Each type of electrode material has specific operating ranges (appropriate charge capacities and voltage ranges for predominantly reversible and limited irreversible reactions) and it can be highly advantageous to operate within those ranges in some embodiments. For example, cathodically biasing titanium nitride and/or tantalum electrodes mitigates formation of oxides that occur with anodic voltages. In another example, anodically biasing titanium develops a protective oxide on the titanium surface that can reduce material corrosion. Cathodic biasing an electrode can protect against corrosion in a similar manner. Any conductive materials (e.g., metals, conductive coatings) exposed to surrounding fluid or electrolyte can be subject to corrosion, and biasing these materials can provide protection against deleterious corrosion to enhance the longevity of service of the active conductive elements of the system. These active conductive elements may comprise any exposed metal on an implantable pulse generator (IPG), for example, that passes current or is subject to voltage potentials that drive undesirable reactions.

In traditional stimulation systems, AC currents are delivered through a series capacitor with no DC leakage to guarantee charge is balanced. However, electrochemical reactions are exponential, time dependent, and not fully symmetrical, so capacitors even at lower voltages do not guarantee elimination of irreversible reactions. Capacitors are an economical way to prevent imperfect (realizable) current sources and sinks from introducing DC and a reliable way of isolating faults caused by current sources and sinks. In some embodiments that can overcome these issues, systems and methods do not include any such capacitors, also known as blocking capacitors.

In some embodiments, chronic electrode materials are biocompatible and do not easily dissolve.

In some embodiments, disclosed herein are systems and methods of stimulation with full spectrum stimulation.

Hybrid waveforms: Conventional stimulation waveforms are intentionally AC and have an unintentional DC component. The unintentional DC component is conventionally kept as low as possible to balance the charge, leaving the voltage equilibrium point not directly controlled. By intentionally injecting or absorbing non-AC current (referred to herein as a bias current), deliberate control of the voltage equilibrium point (quasi-steady state) and range can be achieved. Such deliberate control can be achieved, for example, via one or more of the following non-limiting examples: (a) in the case of monopolar bias current, e.g., DC Bias or similar current injection or absorption can occur by creating an imbalance on one or more WE (working electrodes) and one or more CE/IE (counter electrodes operating as the Indifferent Electrode) without current control that will absorb all of the current, (b) in the case of bipolar bias current, e.g., DC Bias or similar current injection or absorption can occur by creating an imbalance on one or more WE (working electrodes) and one or more CE (counter electrodes) where the excess current will be absorbed on the Indifferent Electrode, (c) and in the case of multipolar some hybrid of (a) and (b). This can create an imbalance on each WE and CE/IE. Bias current or similar current injection or absorption can occur by creating an imbalance between N number of CEs and M number of WEs and absorbing the difference with at least one indifferent electrode, which may or may not be the same as the CE (e.g., source and sink are imbalanced on multiple current outputs); during bipolar operation between two contacts, the WE and CE change between the two contacts depending on the system polarity and the indifferent electrode or electrodes (IE) represents a third electrode type which absorbs the imbalance between the WE and CE regardless of polarity. In some embodiments, a system or method can include only a single WE and a plurality of CEs to advantageously reduce the stress and driving voltages on electrodes. In some embodiments, two WEs can be operated together for a given collective current output to reduce the driving voltages of the two WEs as compared to having the same current output on a single WE.

DC or similar charge injection: Deliberate control of the voltage equilibrium can be utilized for the purpose of adjusting electrode potentials (quasi-steady state voltage), and can be achieved using, for example, a generalized proportional integral derivative (PID) control algorithm where the process variable (PV) can be some environmental measure including but not limited to: a maximum peak voltage; a minimum peak voltage; a combination of a specified voltage rail and peak-to-peak voltage; average voltage levels; root-mean-square; running average; absolute voltage change; rate of voltage change; access resistance; electrical property of the electrode (e.g., capacitance); charge (e.g., accumulation of current over time); and/or chemical measurement, such as pH.

A bias current, e.g., DC or similar charge injection waveform can be derived as a function of electrode material, frequency composition of waveform, pH, local perfusion, tissue type, the type of tissue, target for stimulation, and/or the distance from target tissue. The waveform can be, for example, any one or more of the following: a constant value; a time-varying value; dynamically adjusted to achieve voltage operating points above based upon an algorithm that may be based on one or more of the following parameters: cathodic current amplitude to anodic current amplitude; duty cycle or ratio of cathodic phase time to anodic phase time; interphase or interperiod interval; overall charge in cathodic or anodic phases; and/or types of waveforms in the cathodic or anodic phase. These items assume a current stimulation source but can also be applicable for a voltage stimulator where voltages are used in place of currents to determine the parameters above.

In some embodiments, multidimensional field shaping can be implemented. In some embodiments, multiple electrodes can be spatially employed to focus energy in targeted localities including the dorsal root entry zone; spinal column; spinothalamic tract; and/or any specific spatial region in and around the stimulating electrodes. In some embodiments, systems and methods can include temporally using frequency and phase shift to vary the field such that the focal point moves with time; and/or recruits different classes and diameters of neurons, axons, or ion channels.

In some embodiments, systems and methods can be configured for voltage and/or pulse clamping. Pulse clamping can provide a method of forcing a voltage on the electrode surface to avoid irreversible chemical reactions. Pulse clamping can include control of the voltage, such as, for example, during an interpulse interval, to control the electrochemistry of the electrode so as to “reset” the electrode or control the electrochemistry to allow for more charge to be injected in the next stimulation cycle.

Clamping can also be utilized to increase charge injection capabilities. Clamping and/or utilizing current limits can limit the recruitment of neural tissue and voltage slew rate on an electrode surface. Current passing through the electrode can be used to assess the current state of the electrode, allowing adaptive control of the stimulus waveform to affect parameters including those mentioned otherwise herein.

In some embodiments, systems and methods can generate waveform shapes, such as defined by B-splines, infinitely differentiable functions, and/or other mathematical functions (e.g., sine waves) to control/limit the rate of voltage and current change to: limit/control the onset of neural recruitment or the opposite; and/or limit or control the electrochemical reactions or the opposite. Asymmetric waveforms can be generated where the duration and amplitude of the cathodic phase is different from the duration and amplitude of the anodic phase. The shape of the waveform may comprise changes in amplitudes over time including a linear and non-linear, monotonic or non-monotonic, increases and decreases in amplitude as well as periods of constant amplitude or zero amplitude. In one embodiment a waveform may comprise a sequence with a current with increasing amplitude, a substantially constant current amplitude, a current with a decreasing amplitude followed by the same sequence of the opposite polarity. The magnitude of the slope of the increasing current amplitude may be greater than, equal to, or lower than the magnitude of the slope of the decreasing current. To mitigate undesired neural activity, the magnitude of the slope of the increasing current amplitude may be configured to be lower than the magnitude of the slope of the decreasing current. Furthermore, the transition regions between increasing, substantially constant, and decreasing current amplitudes may be smoothed and rounded to control/limit the rate of voltage and current change to limit/control the onset of neural recruitment. In some embodiments, the first derivative of the waveform current with respect to time comprises a continuous function over time without discontinuities. In further embodiments, the second derivative of the waveform current with respect to time comprises a continuous function over time.

In some embodiments, an amplitude waveform can have various rise time/durations, plateau time/durations, and/or fall time/duration depending on the desired clinical result.

In some embodiments, a waveform can include a rise time of about, at least about, or no more than about 0.25 s, 0.5 s, 1.0 s, 1.5 s, 2.0 s, 2.5 s, 3.0 s, 3.5 s, 4.0 s, 4.5 s, 5.0 s, 6 s, 7 s, 8 s, 9 s, 10 s, 15 s, 20 s, or more or less, or ranges including any two of the foregoing values. The rise time could be a single continuous rise, or multiple rises interspersed with one or more plateau and/or falls in some embodiments.

In some embodiments, a waveform can include a plateau time of about, at least about, or no more than about 0 s, 0.5 s, 1.0 s, 1.5 s, 2.0 s, 2.5 s, 3.0 s, 3.5 s, 4.0 s, 4.5 s, 5.0 s, 6 s, 7 s, 8 s, 9 s, 10 s, 15 s, 20 s, or more or less, or ranges including any two of the foregoing values. The plateau time could be a single continuous plateau, or multiple plateaus interspersed with one or more rises and/or falls in some embodiments.

In some embodiments, a waveform can include a fall time of about, at least about, or no more than about 0.25 s, 0.5 s, 1.0 s, 1.5 s, 2.0 s, 2.5 s, 3.0 s, 3.5 s, 4.0 s, 4.5 s, 5.0 s, 6 s, 7 s, 8 s, 9 s, 10 s, 15 s, 20 s, or more or less, or ranges including any two of the foregoing values. The fall time could be a single continuous fall, or multiple falls interspersed with one or more plateaus and/or rises in some embodiments.

In some embodiments, the waveform starts cathodic first and falls, plateaus, then rises then the waveform becomes anodic and rises, plateaus, and then falls for a set time period. In some embodiments, the waveform starts anodic first. The set time period can be, for example, about, at least about, or no more than about 1 s, 2 s, 3 s, 4 s, 5 s, 6 s, 7 s, 8 s, 9 s, 10 s. 11 s, 12 s, 13 s, 14 s, 15 s, 16 s, 17 s, 18 s, 19 s, 20 s, 25 s, 30 s, 35 s, 40 s, 45 s, 50 s, 55 s, 60 s, or more or less, or ranges including any two of the foregoing values. In some embodiments, the cathodic and anodic phases are separated by a third plateau type.

In some embodiments, waveform charge per phase (e.g., amplitude, waveform shape and/or pulse-width) can be controlled to selectively modulate and recruit neurons of differing diameter, conduction speed, and type (e.g., myelinated and unmyelinated).

In some embodiments, systems and methods can incorporate a variety of waveform frequencies, including high frequencies, e.g., about 1.2 kHz-50 kHz or higher; conventional frequencies, e.g., between about 20 Hz-1.2 kHz; low frequencies, e.g., between about 1 Hz-20 Hz; and ultra-low frequencies, e.g., below about 1 Hz. As noted elsewhere herein, direct current as defined herein is inclusive of low frequency and ultra-low frequency AC current waveforms that are perceived as and functionally are direct current from the perspective of the tissue whose action potentials are being modulated.

A signal that averages a non-zero current over the duration of stimulation can include, for example, a charge imbalance, including but not limited to an intraphase bias, phase-to-phase bias, a cycle-to-cycle bias, an intermittent bias or offset, or any combination thereof. The bias may also be configured to be time-varying independent of the stimulation waveform.

In some embodiments, systems and methods can include pre- or post-conditioning before or after quasi steady-state stimulation. This can be important to, for example, limiting or ramping the voltage change rate so as not to invoke undesired electrode chemical reactions; slowly transitioning voltages or currents between on and off states to limit neural recruitment changes that can result in patient discomfort or off-target effects; and/or manage charge on, or voltage across, the electrode surface by effectively time shifting especially in the case of bipolar/multipolar where it can be desirable that waveforms on each electrode start and stop on cathodic cycles. Pre/post conditioning can allow an effective time-shift and ramp.

In some embodiments, systems and methods can include in vitro or in vivo preconditioning of electrodes using cyclic voltammetry or electroplating techniques. These can be used, for example, to remove electrode material defects in a controlled fashion to not overstress defect areas with high current densities and cause expansion rather than removal of defects. For example, a sharp point or edge on the surface of an electrode can generally have a higher current density relative to the rest of the electrode surface. This may lead to metal dissolution and formation of undesirable chemical species that may in that particular area accelerate electrode degradation. By etching away sharp edges in a controlled fashion, e.g., in vitro, the process can be directly controlled. Preconditioning can also be utilized for material deposition or controlled electrochemical conversion (e.g., formation of a protective oxide layer). Oxide growth can also be used to increase the charge capacity of the material, e.g., activated iridium oxide (AIROF) as one example.

Some embodiments can utilize AC and/or DC neuromodulation (e.g., stimulation or block) signals. AC signals can be used, for example, to include action potentials; paresthesia; and/or disable neurons to block neural signals.

In some embodiments, DC (including ultra-low frequency stimulation) can be utilized in addition to blocking action potentials to allow for superposition of an electric field across neurons for the purposes of inducing a transmembrane potential that can allow modulation of one or more of the following neuronal properties: Spiking properties; Excitability (increase or inhibit sensitivity); Conduction velocity; Control of electroporation; synchronization of neural populations; induction of secondary effects in cells that affect neighboring cells including vascular changes and blood brain barrier permeability; shift strength duration curve; DNA or RNA transcription or translation modification; and/or Protein or drug transport (electrophoresis).

In some embodiments, the electrode (working electrode, indifferent electrode, or other electrode) can comprise material(s) configured to optimize the charge capacity, durability, and biocompatibility, including, for example, any of: Iridium Oxide; Tantalum, MP35N, Titanium Nitride; Diamond; Boron-doped diamond; Nanotubes (e.g., TiN, MnO₂/TiN); Metal Oxides (e.g., RuO₂, IrO₂, MnO₂, CoO₂, NiO₂, FeO₂, SnO₂ and CuO₂); Mixed Metal Oxides (e.g., RuO₂/SnO, RuO₂/NiO, RuO₂/Ta₂O₅, RuO₂/Pt, RuO₂/TiO₂. RuO₂/MoO₃, RuO₂/CaO and RuO₂/V₂O₄ or IrRu); Carbon/Graphene; metal carbides, carbonitrides and nitrides aka MXenes (or transition metal carbides, carbonitrides and nitrides); and/or combinations of any of the foregoing (including two or more metal oxides; two or more mixed metal oxides, a first material with a coating including the first material, and/or a second material, etc.).

In some embodiments, to increase the available electrochemical surface area (ESA), the substrate of an electrode may comprise microstructural features that increase the available surface area. In some embodiments, the substrate may comprise an open porosity sintered component. In some embodiments, the substrate may comprise an open porosity foam component such as a reticulate foam structure. In some embodiments, the substrate may comprise surface texturing from micromachining to generate features such as grooves or roughness to increase the overall ESA. These micromachining tools may comprise laser ablation to generate channels or grooves or general surface roughness. Additional micromachining techniques which can accomplish these goals are electric discharge machining (EDM), material etching techniques, pattern masking and etching techniques, bead or grit blasting, and surface sanding. The ESA can also be increased by, for example, electrophoretic deposition (EPD); electrolytic deposition (ELD); and/or electroplating. The substrate material can, for example, be coated with a high charge capacity material such as sputtered iridium oxide (SIROF), Poly(3,4-ethylenedioxythiophenc)-poly(styrenesulfonate) (PEDOT), titanium nitride (TiN), fractal titanium nitride, porous titanium nitride, or some combination thereof. Methods for depositing such coatings on compatible substrates may include chemical vapor deposition (CVD) and physical vapor deposition (PVD). Substrates may comprise electrically conductive materials to allow for ease of charge transfer to the coating.

In some embodiments, systems and methods can include a physical set of electrodes and the pathways of which the stimulation can be delivered. Borrowing from electrochemical cell terminology, in some embodiments, a system can include N working electrodes (WE) tied to individual lead electrode contacts, one or more Indifferent Electrodes (IE), and optionally one or more Reference Electrodes (REF) if desired to measure half-cell potentials. The counter electrode (CE) can either be assigned to a WE or IE depending on the stimulation modality.

In some embodiments, measurement of electrode potentials can include voltages across any two of the electrodes (e.g., WE, CE, IE and REF) when stimulated with a known current. These measurements can be used to measure and track characteristics of the electrode including the impedance and capacitance of the electrode statically or dynamically during stimulation to assess the condition of each electrode and the system in general.

In some embodiments, any unstimulated electrode (e.g., electrode on the lead) may be assigned to operate as a REF and measure voltages/impedances based upon physical location to image and characterize physiological tissue. Any number of, or all of the electrodes can be switched between states, e.g., a reference electrode can be transformed into a working electrode, and a working electrode can be transformed into a reference electrode. Such transforms may based on the phase of the stimulation pulse, whether current is being transmitted through the electrode or not, or some other factor. In some embodiments, the WE or REF can be interchangeable depending on necessary current delivery (e.g., electrical conduction block with one segment, e.g., half of lead and a subsequently created block with another segment, e.g., half of lead to target different regions, and other examples). When using a WE-indifferent electrode pair, with DC current through IE at steady state, WE changes can be monitored. This configuration may be used to monitor the system and/or for adaptive control.

For example, the voltage on an electrode may indicate what electrochemical reactions are going to occur. For example, if the voltage is outside of the water window for the electrode (the window varying based upon the material of the electrode), then water may break down into H₂ and O₂, which can be damaging to neural tissue or the electrode. Furthermore, if the voltage causes a reaction associated with corrosion, then electrode corrosion and deterioration may occur. If the voltage is in either these ranges where these or other faradaic electrochemical reaction occurs, it may be desirable to permanently or tentatively cease therapy, such as, for example, by stopping operation of the external pulse generator 106. Water windows and anti-corrosion ranges are discussed further below with respect to FIG. 11.

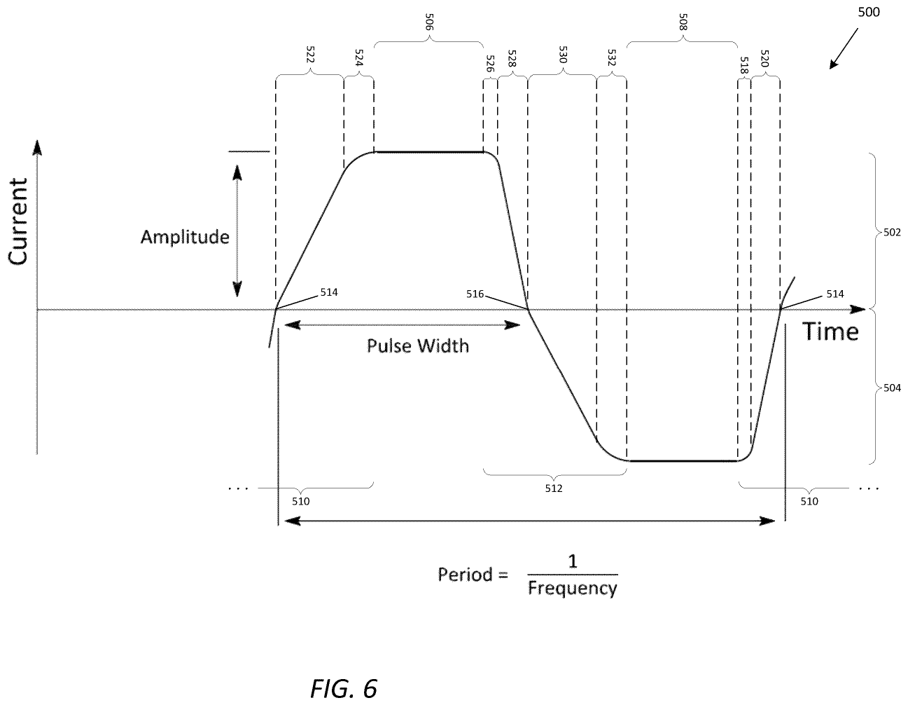

The voltage on a working electrode is monitored to determine whether the system is operating in the desired voltage range. The relative or absolute voltage, or changes to either, or combinations, may be monitored. Relative voltage corresponds to the peak-to-peak voltage of the waveform and/or its subcomponents. If the relative voltage is out of range, the lead electrode may decay, which will increase capacitance, which will cause the magnitude of the peak-to-peak voltage to further increase. The peak-to-peak voltage may be determined as a difference between the maximum voltage and the minimum voltage, an average of peak-to-peak voltages, or a ratio of peak-to-peak voltages, for example, during anodic and cathodic phases of the output waveform (e.g., waveform 500 of FIG. 6). The relative voltage may be measured between a working electrode and a reference electrode, or between a working electrode and an indifferent electrode. Such peak-to-peak voltages are preferably taken once the system has reached a steady state, for example, a steady state voltage on the indifferent electrode.

Absolute voltage may be monitored in addition to or instead of a relative voltage. For example, if the absolute voltage drops below a threshold level, water may break down (e.g., outside of the water window) and destroy tissue and/or other deleterious reactions may occur. If the voltage exceeds a threshold level, water may break down and destroy tissue and/or corrosion or dissolution of the electrode metal may occur, as well.

In some embodiments, stimulation modalities include the following: monopolar where the current is driven through a lead electrode contact (WE) with positive and negative pulses of current and sinked by one or more indifferent electrodes; DC or similar current injection (bias) affected by shifting the waveform positive or negative; Bipolar where the current is driven with opposite polarities onto a pair of lead electrode contacts operating alternatively in the WE and CE roles, and any imbalance will be absorbed by one or more IEs; DC current injection (bias) affected by shifting each waveform positively or negatively and the difference can be absorbed by the IE(s); multipolar where the current is driven with opposite polarities onto a set of lead electrode contacts operating alternatively in the WE and CE roles. Any imbalance will be absorbed by the IE(s); and/or DC or similar current injection (bias) affected by shifting each waveform positively or negatively and the difference can be absorbed by the IE. Stimulation current outputs can be realized in some embodiments with the following mechanisms: Bipolar power supplies where current sources are driven by the positive supply and current sinks are driven by the negative supply providing unconstrained simultaneous positive and negative currents and the IE is tied to the Ground (0 V); and/or a monopolar power supply where a current source and sink are put in series and a virtual ground develops between the two supplies.

In some embodiments, directly controlling electrode surface voltages during simultaneous stimulation can require additional techniques since the interaction between all the elements is related. This can be addressed, for example, in the following ways: In the case of simultaneous stimulation periodically turning off current to all but one electrode pair and making measurements at that time; Shifting phase between electrode pairs and making measurements when other pair(s) crosses zero current; utilizing reference electrodes located in proximity to the driven electrodes to that are to be measured; Limiting using worst-case voltages from the electronic current sources at the expense of not deriving all the performance of each electrode; control using voltages rather than current sources (voltage clamping), and/or control by using injected currents rather than voltages (bias currents).

In some embodiments, systems and methods can include electrode restoration techniques, including but not limited to Periodic Restoration-drive voltage/current to reset electrochemical surfaces, e.g., by periodically delivering a cathodic charge to drive down electrode voltage; and/or Adaptive Restoration-cycle by cycle drive voltage/current to preserve electrochemical surfaces, e.g., by dynamically adjusting the charge driven through the electrode to control electrode voltage.

In some embodiments, compliance voltage limiting can be utilized as an automatic way to limit chemical reactions (e.g., by setting compliance voltage such that there is enough voltage to drive the current sources and safe chemical reactions, but not enough voltage to provide electrode voltages sufficient to drive undesired reactions). In some embodiments, compliance voltage tracking can be utilized to reduce power dissipation. Current sources and sinks can be used to control current, and a switching variable power supply can generate just enough voltage to keep current sources and sinks in compliance, e.g., to supply the requested current. An alternate topology can be a variable voltage source to drive a specified current. One advantage of compliance tracking is that the current sources using MOSFET gate ratios (e.g., Wilson current sources) are inherently stable.

In some embodiments, driving voltage levels can be limited between pairs of electrodes as a means to limit chemical reactions or stop an electrode that is degrading and requires a higher driving voltage to deliver the target stimulation current.

In some embodiments, high frequency stimulation (e.g., high frequency alternating current or HFAC stimulation) can be biased with a low frequency signal to reduce onset effects. For example, a low frequency signal (sometimes referred to herein as a DC, low frequency DC, or ultra-low frequency (ULF) signal) may be provided before an HFAC signal, concurrent with an HFAC signal, constantly with HFAC, or only during onset and/or offset with HFAC.

In some embodiments, electrodes may comprise materials on which reversible electrochemical reactions can occur such as platinum which forms a hydride complex on its surface in aqueous solutions. To generate more surface area for reversible electrochemical reactions to occur, traditional electrodes and electrode materials may be made from high surface area to volume structures such as electroplated surfaces, (e.g., platinum black/electrodeposited iridium oxide for example), roughened surfaces, woven surfaces, patterned surfaces, reticulated foam structures, porous sintered bead structures, nano- or micro-patterned structures to expose additional material surface area. High-charge chemistry electrodes can be biocompatible, or suitably sequestered from body tissue if not. In some embodiments, the electrode driving current may be between about 0 mA and about 1 mA, between about 1 mA and about 2 mA, between about 2 mA and about 4 mA, between about 4 mA and about 8 mA, higher than about 8 mA, about 0.5 mA, 1 mA, 2 mA, 3 mA, 4 mA, 5 mA, 6 mA, 7 mA, 8 mA, 9 mA, 10 mA, or ranges incorporating any two of the foregoing values. In some embodiments this driving current is then used to generate a corresponding ionic current of similar magnitude, depending on the specific reversible electrochemical reactions.

Some embodiments can include a material such as tantalum or titanium nitride to generate a capacitive traditional electrode interface instead of an interface at which an electrochemical reaction occurs. Transparent conducting oxides (TCOs) such as fluorine-doped tin oxide (FTO), nickel titanium dioxide (Ni/TiO2), and other titanium dioxide (TiO2) constructs are also candidate materials that have high charge carrying capacities, as well as others disclosed herein. In some embodiments, the electrodes can include tantalum coated with titanium nitride. In some embodiments, the electrodes can include MP35N coated with titanium nitride. In some configurations, charge generation at the traditional electrode surface would attract ionic species from the ionically conductive material until the charge at the traditional electrode interface is passivated. Charging of the capacitive material with an electric current of one polarity can generate current flow in the form of ions. Reversing the polarity of the current flow to the capacitive material can effectively reset the system for a subsequent charging to generate further ionic current flow. To generate more surface area for increased ion current flow capacity to occur, traditional electrodes may be made from high surface area to volume structures such as roughened surfaces, reticulated foam structures, porous sintered bead structures, nano- or micro-patterned structures to expose additional material surface area. In one embodiment, this capacitive structure is in fluid contact with an electrolyte solution that contacts an electrolyte-saturated hydrogel in contact with target nerve tissue to enable ion current flow to the tissue. In some embodiments, the solution is body fluid such as interstitial fluid that delivers the current to the electrically excitable tissue from the electrode.

To deliver current to the nerve to facilitate a block, the electrode may be connected via a conductive lead to one or more current sources. A single nerve-electrode interface can provide nerve block when current is applied in one polarity (blocking phase). When the current polarity is reversed to return the electrode to its original state (which may be a non-blocking phase or also a blocking phase), the nerve may or may not continue to block pain stimulus from passing along the nerve. If the nerve has been placed into a state of hypersuppression, the nerve will continue to prevent action potential propagation and block pain signals regardless of the phase state of the electrode. However, if the nerve is not placed into a state of hypersuppression, the nerve may pass undesirable signals when the blocking-phase signal in absent. Fridman and Santina have described a means to enable continuous block when current polarity is reversed such that the neural tissue see a constant current while each driving electrode experiences an alternating current (AC) using a series of valves to direct current flow direction (Fridman and Santina, Safe Direct Current Stimulation to Expand Capabilities of Neural Prostheses, IEEE Transaction of Neural Systems and Rehabilitation Engineering. 2013 Mar. 21(2):319-328; Fridman and Santina, Safe Direct Current Stimulator 2: Concept and Design, Conf Proc IEEE Eng Med Bio Soc, 2013: 3126-3129). However, in some cases it is desirable to have a simpler system which does not require the use of valves that present additional failure points and add bulk to an implantable system. A simpler, more robust system may be configured without valves and such moving parts by using multiple electrodes to provide constant stimulation of the nerve tissue itself. In one embodiment to provide continuous block, two nerve-electrode interfaces are present and connected to one or more current sources. The first nerve-electrode interface electrode is run with the current in one polarity to drive a block while the second nerve-electrode interface is run with the opposite polarity. After a period of time, the current polarities of the first and second electrodes are reversed, and the second nerve-electrode interface provides a block while the first nerve-electrode state is reversed to its prior state. By cycling the electrode currents, a continuous block can be maintained at the target nerve. As can be appreciated, more than two, such as 3, 4, 5, 6, 7, 8, 9, 10, or more electrodes may also be used to facilitate the same continuous block. Electrodes may also be run in either monopolar or bipolar configurations. In some embodiments the electrodes system is configured to not have any actuated mechanically moving parts, such as valves or hinges.

Alternatively, nerve activity may be suppressed, which means that nerve activity remains blocked or reduced even after removal or discontinuation of the blocking current. The nerve may be further put into a state of hypersuppression in which the nerve remains blocked or reduced in activity without rapid reversibility after cessation of DC delivery. Modulation of the initial current delivered to the nerve tissue including ramp rate, current amplitude, total charge delivery, and waveform shape can be used to place the nerve in a state of suppression. During the state of suppression, the electrodes may be returned to its initial state by reversing the current polarity used to generate the initial block and suppression state. During the period of reverse current flow, the nerve may remain in a state of hypersuppression. In another configuration the system may deliver subsequent blocking or activity reducing current inputs that extend the suppression duration, with periods of no current delivery, or of reversal current in between current doses. The nerve tissue may remain in a state of hypersuppression during the periods of non-blocking or non-activity reducing current input. In another configuration, the system may be configured to deliver subsequent current inputs on a schedule. In some embodiments, the DC blocking or activity reducing waveform may have an amplitude of between 0-250 microamps, 250-500 microamps, 500-1000 microamps, 1000-1500 microamps, or 2000 microamps, or higher, or about, at least about, or no more than about 50, 100, 150, 200, 300, 400, 500, 600, 700, 800, 900, 1,000, 1,100, 1,200, 1,300, 1,400, 1,500, 1,600, 1,700, 1800, 1,900, 2,000, 2,500, 3,000, 3,500, 4,000, 5,000 microamps or more, or other ranges incorporating any two of the aforementioned values. Placing a nerve into a state of hypersuppression may be facilitated in some embodiments by delivering a charge of 1-2 millicoulombs, 2-5 millicoulombs, 5-10 millicoulombs, 10-50 millicoulombs, 50-100 millicoulombs, 100-500 millicoulombs, 500-1000 millicoulombs, or 1000 millicoulombs or greater, or about, at least about, or no more than about 1, 2, 5, 10, 25, 50, 100, 200, 300, 400, 500, 600, 700, 800, 900, 1,000, or more millicoulombs, or other ranges incorporating any two of the aforementioned values, and depending on nerve size and desired hypersuppression duration. Activity reducing amplitude and current duration may be tuned to enable hypersuppression in the range of, for example, 0-0.5 times the duration of initial blocking or activity reducing waveform delivery, 0.5-1 times the duration of initial waveform delivery, 1-1.5 times the duration of initial waveform delivery, 1.5-2 times the duration of initial waveform delivery, and greater than 2 times the duration of initial waveform delivery, or about, at least about, or no more than about 0.1×, 0.2×, 0.3×, 0.4×, 0.5×, 0.6×, 0.7×, 0.8×, 0.9×, 1×, 1.1×, 1.2×, 1.3×, 1.4×, 1.5×, 1.6×, 1.7×, 1.8×, 1.9×, 2×, 2.5×, 3×, 4×, 5×, or more relative to the duration of initial waveform delivery, or ranges including any two of the aforementioned values.

Sensing the local state of and proximal to the nerve tissue can also provide a useful measure for determining when to provide current inputs to extend nerve suppression as well as to provide a feedback loop for initial current delivery to generate the initial nerve block by modulating the nerve potential such that it cannot transmit action potentials. In one embodiment the nerve's ability to conduct action potentials is monitored such that as direct current is delivered to the nerve tissue, the direct current delivery can be maintained to ensure that the nerve block is maintained, for example. Nerve conduction ability may be monitored by any suitable measure such as delivering a stimulus pulse and measuring compound action potential signals.

In some embodiments sensing is in the form of a reference electrode to measure potential differences relative to the two electrodes which are passing the active current. In some embodiments the active current is modulated in response to one or more measured electrode potentials relative to the reference electrode. In some embodiments the active current is modulated when measured electrode potential indicates that undesired electrochemical reactions may occur at one or more active electrodes. For example, active current may be reduced or ceased upon measurement of an active electrode potential that indicates water electrolysis is occurring or possible. The system may be operated with a direct current input or by applying a potential difference between the working electrode and an auxiliary or indifferent electrode. In some embodiments, a reference electrode may be located within the system or at the distal end of the system proximal to the nerve tissue.

Using these methods of placement of blocking or activity reducing electrodes along the spinal column to modulate activity in the spinothalamic tract and the ability to tune the electric field to generate nerve block or activity reduction and/or suppression, specific targets for pain attenuation can be facilitated. For example, trunk pain which is moderated by the thoracic vertebral levels can be modulated by placing leads along the thoracic spine while neck pain may be moderated by providing block or activity reduction and/or suppression in the cervical spine. Upper limb pain may be moderated by providing a combination of cervical and thoracic level block or activity reduction and/or suppression while lower limb pain may be moderated by a combination of lumbar and sacral level block or activity reduction and/or suppression in the spine.

Generation of pain block or attenuation can be used to facilitate peri-procedural pain block where motor control and non-pain sensations are desired. For example, in labor and delivery of a child, one of the challenges with pain management particularly with epidural anesthesia is the reduction in ability to be sensate in the lower body. Due to the non-specific nature of the delivered anesthesia in the epidural space sensory, pain, and motor neurons are impacted. The epidural anesthesia can lead to difficulty with generating pushing force during the birthing process and can lead to numbness a few hours after birth impairing motor abilities such as the ability to walk. In some instances, epidurals are further implicated in fetal and newborn health including breastfeeding difficulty. Using the blocking electrodes described above to target the spinothalamic tract and/or dorsal root ganglia, the undesired pain can be targeted without generating the side effects (or reducing side effects) associated with current epidural anesthesia techniques because only the pain tracts are targeted and not any other motor or sensory tracts. Furthermore, in the case in which ionic current is delivered to the nerve tissue in a reversible blocking fashion, the stopping of block can enable the patient to immediately be restored to normal pain sensation if desired and any off-target block can be reversed enabling immediate body function restoration.

Beyond central nervous system interventions, a safe direct current block or activity reduction can also be facilitated in the peripheral nervous system in which electrodes are placed in contact or in proximity to peripheral nerves to facilitate block or activity reduction. Specific pain targets include, for example, focal pain, phantom limb pain, neuroma pain, and neuralgias. A pain target could also include post-operative pain. Targeting the peripheral nerves proximally (i.e., closer to the spinal cord) from the site of pain for block or activity reduction can suppress pain from the distal site. Specific to neuralgias, postherpetic neuralgia (after shingles) can be targeted based on the presentation of the outbreak which will trace specific dermatomes. For trigeminal neuralgia, the trigeminal nerve (and/or trigeminal ganglion and/or trigeminal nucleus in the brainstem) can be targeted for block to reduce pain that commonly manifests as facial pain. Another target is the glossopharyngeal nerve which produces pain in the neck and throat. Neuralgia in extremities such as the hands, arms, feet, and legs as frequently caused due to diabetes-related neuropathies are also potential targets.

In some embodiments, electrical neuromodulation of tissue including delivery of high charge densities via an ultra-low frequency waveform and use of at least one indifferent electrode to absorb a bias current can unexpectedly, advantageously, and safely enable continued suppression of neural activity after cessation of current delivery (wash-out period), which can provide multiple benefits. For example, power can be saved or, stated differently, power consumption can be slowed. In another example, the sudden return of neural activity can be prevented in case of device failure (e.g., connection, battery, etc.), which can improve safety and/or enable patient/physician intervention during prolonged suppression period. Similarly, the rapid return of pain in the event of device failure and/or the rapid return of sympathetic signaling that may lead to acute sympathetic events (such as acute decompensation or cardiac arrhythmias) can be prevented. In yet another example, when implemented as an electronic medicine dosing system/method, a long-lasting therapeutic benefit can be created from acute application. Furthermore, such systems and methods can be very safe, inhibiting/minimizing the creation of toxic species at the electrode-nerve interface.

In some embodiments a system is configured for generation of nerve block or activity reduction for disorders and diseases that can be addressed by reducing neural activity in specific regions of the brain responsible for the specific disorder. Neural activity reduction can be facilitated by directly blocking and/or reducing activity of specific neurons as well as by blocking pathways along which excessive neural signaling is occurring. In some embodiments, this system for deep brain block (DBB) comprises all or some of the steps of identification of the anatomic target site for block, creating an access site to the exterior of the brain tissue, creating a path through the brain tissue to the target site, evaluating the suitability of the target site for block, adjusting or refining the location of the target site, providing nerve block at the target site, and adjusting the nerve tissue block strength or location. Practically, this process may be implemented using techniques known in the field of deep brain stimulation (DBS) in which a target anatomic site is identified using a combination of imaging techniques such as but not limited to magnetic resonance imaging (MRI) including functional MRI (fMRI), computed tomography (CT), PET scanning, and/or x-rays. This site can then be accessed using stereotactic techniques to register an identified region from imaging to the physical anatomy on the patient. A frame may be fixed to the patient's head and skull to allow for spatial registration during the procedure. An access site to the brain tissue in the form of a burr hole or craniotomy can be formed with or without additional access tools fixed to the skull such as insertion cannula and advancement/retraction equipment to access the target site. Advancement of a nerve tissue activity measurement probe through the brain tissue to the target site may be used to enable evaluation of the suitability of the brain region. This probe may record neural activity to determine that the measured signals are consistent with that of tissue requiring block or activity reduction. If the signal characteristics indicate that the location is not optimal or appropriate for block or activity reduction, the probe may be adjusted until the correct location is identified. The measurement probe may be exchanged with the therapeutic electrode which can then be inserted with the active portion of the electrode positioned within the target site. Activation of the blocking or activity reducing signal can then be used to assess efficacy of the block as well as to tune the strength of the signal. The therapeutic electrode can then be fixed to the skull to maintain the active portion's (e.g., region delivering ionic current) position at the target site. An extension lead can be connected to the affixed therapeutic electrode and connected to an implantable current source, similar to an implantable pulse generator (IPG), whose output signal can be adjusted to facilitate optimal symptom reduction. Blocking or activity reducing electrodes may be implanted unilaterally or bilaterally as the contralateral side of the body is affected by specific anatomic target sites.

Disclosed herein in some embodiments are systems and methods to limit undesired electrochemical reactions by monitoring signals indicative of conditions favorable for such reactions to occur and modification of the reaction generation conditions. Not to be limited by theory, traditional alternating current stimulation of neural tissue typically delivers a relatively low amount of charge through conventional electrodes (e.g., platinum electrodes). However, in some embodiments, high charge density electrodes including those described elsewhere herein deliver relatively greater amounts of charge closer to and beyond, and in some cases far beyond, the Shannon limit. Control systems and methods such as those disclosed herein can surprisingly and advantageously allow for the safe delivery of such current to tissue.

In some embodiments, a system can include a monitoring system, e.g., including a hardware and/or software controller configured to measure the voltage required to generate the electric current to drive the stimulation current. If the voltage crosses a threshold, e.g., a predetermined threshold, the controller can adjust (e.g., increase or decrease) the current output to bring the voltage level into an acceptable range relative to the threshold voltage level. For example, if the voltage required to maintain a specific current level becomes too high, the current level may be reduced to the point that the voltage falls below the defined threshold. In some embodiments, if the voltage required to maintain a specific current level becomes too high, the current level may be set to zero. In yet another alternative system, if the voltage required to maintain a specific current level becomes too high the current level may be reversed.

In some embodiments, the current may take the form of a waveform such as, for example, a square wave or other arbitrary shape in which current is passed between the two electrodes with opposite polarity. The voltage waveform required to drive the current between the electrodes may fall within an upper and lower voltage threshold limit. Over time, if the underlying electrode charge capacity is found to change because of various conditions, the driving voltage waveform required to maintain the target current waveform may also change. If the excursions/deviations from the target thresholds are significant enough, this may be indicative of undesired electrochemical reactions occurring. The voltage threshold limits and associated voltages may alternatively or additionally be measured between the working electrode and reference electrode to directly assess the voltage drop across the working electrode-electrolyte interface to assess the propensity for undesirable electrochemical reactions and voltage potentials across that interface. In some embodiments the voltages between two or more working electrodes may be used to assess electrode status. In some embodiments the voltages between a working electrode and indifferent electrode may be used to assess electrode status. To prevent these excursions into undesirable zones, the current delivered may be adjusted to reduce the driving voltages as described above. Alternatively, the voltage excursions may be due to changes in electrochemistry. For example, in a system in which a reaction occurs where the same target amount of charge is transferred from one electrode to a second electrode and back to the first electrode, the net charge over time may drift from zero (e.g., become unbalanced) due to imperfect charge accounting. This in turn may lead to changes in voltage required to generate the desired current and be indicative of undesired electrochemical reactions occurring. The drift in net charge transfer from a target level may be countered in some embodiments by monitoring the driving voltage and generating a control loop that generates additional charge on an electrode which has been detected via its driving voltage characteristics to be deficient in reactants. In other embodiments a bias current may be used to explicitly unbalance the charge transfer and drive the electrode operating voltage into a more cathodic or more anodic state.

In some embodiments, the electrode could have a working surface area of, for example, about, less than about, or no more than about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 90, 100 mm², or more or less, or ranges including any two of the foregoing values.

In some embodiments, the maximum electrode voltage limit could be, for example, about, less than about, or no more than about −0.3, −0.2, −0.1, 0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 2.5, 3, 3.5, 4, 4.5, 5 V, or more or less, or ranges including any two of the foregoing values.

In some embodiments, the peak-to-peak voltage limit could be, for example, about, less than about, or no more than about 500, 600, 700, 800, 900, 1000, 1100, 1200, 1300, 1400, 1500, 2000, 2500, 3000, 4000, 5000, 10000 mV or more or less, or ranges including any two of the foregoing values.

In some embodiments, the voltage variation limit could be, for example, about, less than about, or no more than about 0.1, 0.5, 1, 5, 10, 15, 20, 25, 50, 75, 100, 125, 150, 175, 200, 225, 250 mV, or more or less, or ranges including any two of the foregoing values. Voltage variations can be measured over durations such as minutes, hours, or days, for example.

In some embodiments, the time period limit could be, for example, about, less than about, or no more than about 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 120, 200, 500, 1000, 5000, 10000 seconds or more or less, or ranges including any two of the foregoing values.

In some embodiments, such as in the algorithms disclosed, for example, the electrode can be conditioned in a manner that allows for delivery of the target current amplitude for the desired duration with a driving voltage below a set threshold such as the electrolysis potential, for example. The algorithm also limits the amount of time the electrode is exposed to voltages above the driving voltage upper threshold which can lead to generation of deleterious by products that can damage tissue.

In some embodiments, direct current (DC) delivery amplitudes for any therapy disclosed herein could be, for example, in the range of about 0 to about 0.5 mA, about 0.5 to about 1.5 mA and about 1.5 to about 2.5 mA, or ranges incorporating any two of the foregoing values. In some embodiments, about 2.5 mA to about 5 mA and about 5 mA, about 5 mA and about 15 mA, and above can be utilized both for anodic and cathodic current levels for block. Response has been seen, for example at about 0-1.5 mA range in animal studies as well as paresthesia onset in peripheral human nerves at about 1.5 mA (cathodic and anodic) with complete block at about 2.5 mA and current delivery up to about 5.5 mA. Therapeutic values for pain relief have been seen in the spinal cord in, for example, the 0.5-1.5 mA range, or about, at least about, or no more than about 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5 mA, or more or less, or ranges including any two of the foregoing values.

In some embodiments, direct current delivery to a patient can be targeted to any number of anatomical locations, including but not limited to: the dorsal root ganglion; dorsal roots; dorsal columns; dorsal horn; Lissauer's tract; and/or the antero-lateral pain tracts. In the spine, the stimulation delivery can be targeted to any number of vertebral levels including but not limited to the sacral region, the lumbar spine, the thoracic spine, the cervical spine as well as over specific spinal disk locations such as, for example, any number of disks between any two of C2, C3, C4, C5, C6, C7, T1, T2, T3, T4, T5, T6, T7, T8, T9, T10, T11, T12, L1, L2, L3, L4, L5, S1, S2, S3, or S4, including but not limited to the disk bridging the T9 and T10 vertebra. In some embodiments, direct current delivery can be directed to a peripheral nerve, or other target locations as described elsewhere herein. Not to be limited by theory, in some embodiments DC delivery can potently modulate small diameter fibers in the spinal cord and depolarize spinal cord neurons. DC delivery may not necessarily be sensitive to fiber size and may have a wide therapeutic window. DC delivery can be utilized for a wide variety of indications, including but not limited to cardiac mapping for arrhythmias, epilepsy, and movement disorders, as well as a variety of other conditions disclosed elsewhere herein.

The systems and methods described herein may be used to generate DC nerve block, modulation or attenuation. Depending on the specific direct current application of nerve block, nerve suppression, or continued block after removal or stopping of the current may occur, and hypersuppression may result for continued nerve blockade in excess of one minute after removal of the DC source to delay nerve conduction recovery. The nerve block and suppression may be generated in an intermittent or continuous manner depending on the desired application. Means for continuous nerve block have been described that provide for safe delivery of nerve block via ionic current utilizing multiple electrodes or sequenced electrode contact activation enabling a means to modulate nerve conduction safely without necessitating complex mechanical systems. The system may be fully or partially implantable, or completely non-implantable (e.g., transcutaneous) with all tissue contacting materials biocompatible for tissue contact and implantation compatibility.

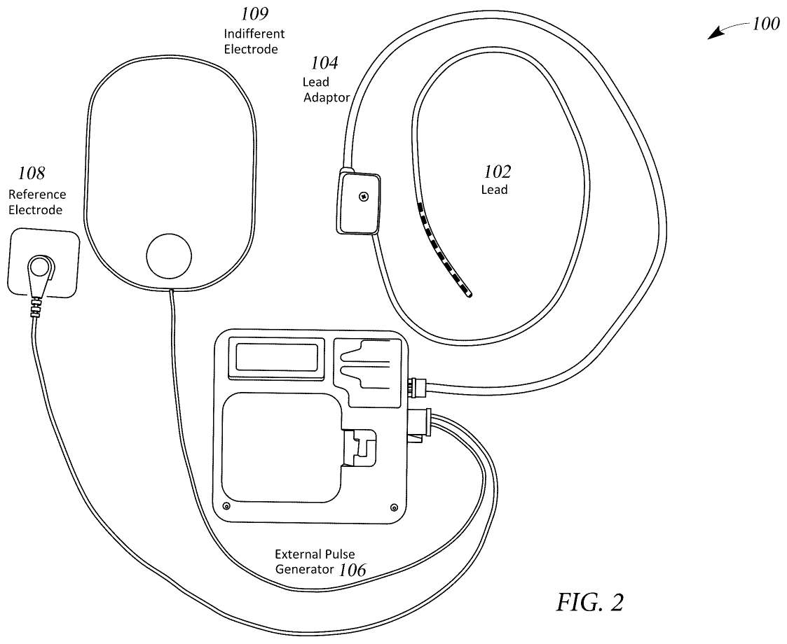

FIG. 2 schematically illustrates one non-limiting example of a neuromodulation system 100. The system can include any number of the following: 1) the Leads (one shown) 102, 2) the Lead Adaptor 104, 3) the External Pulse Generator 106, 4) the Clinician Programming Application (CPA)/Clinician Programming Device (CPD) (not shown); and 5) the Surface Electrodes (optional Reference Electrode 108 and Indifferent Electrode 109) 108, 109.

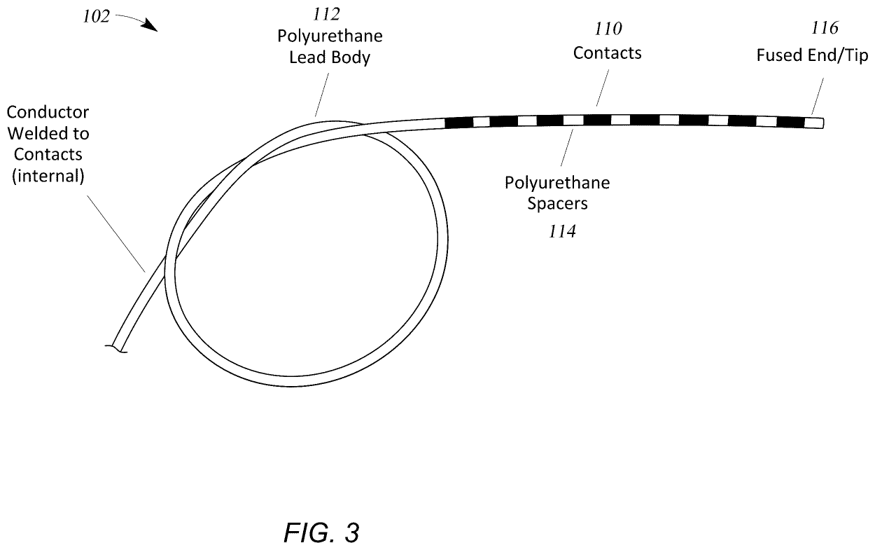

FIG. 3 schematically illustrates an embodiment of a lead 102, which can be, for example, an epidural lead. In some embodiments, a lead 102 could have, for example, a diameter of between about 1 mm and about 2 mm, and can be an appropriate geometry, e.g., cylindrical in shape with up to eight individual contacts (or more) 110 occupying the lead's distal working end. The lead 102 may be, for example, 40-100 cm in length in increments of 5 cm or more or less in length. The individual contacts 110 can be made of a substrate coated with a high-capacity charge coating, including but not limited to titanium coated with titanium nitride, tantalum coated with titanium nitride, platinum-iridium coated with titanium nitride or other materials as disclosed herein for high-capacity current delivery. Each contact 110 may be welded to a conductor which may comprise, for example, platinum-iridium, MP35N, 35N LT, stainless steel: 304SS, 316LVM, or titanium wire which terminates in a connector. The conductors may also terminate in ring-style connector contacts such as Bal Seal style connectors. The lead body 112 itself can be made of medical-grade polyurethane. The lead 102 can include multiple lumens, at least one for each wire and at least one central lumen for a stylet, which may be used for steering and increasing the column strength of the lead 102 during insertion. One or more spacers 114, which can include polyurethane spacers (or other electrical insulative material), can be positioned between adjacent contacts 110. The lead 102 can terminate in a tip 116, which can be a fused end.

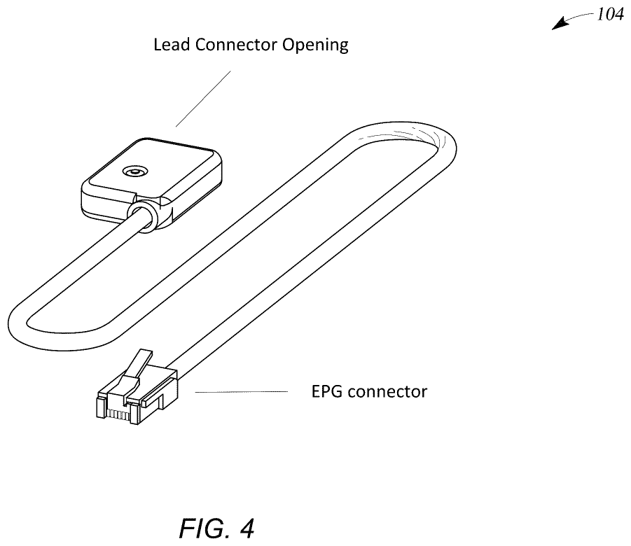

FIG. 4 schematically illustrates a lead adaptor 104, according to some embodiments. The lead adaptor 104 mates the lead 102 (not shown) to the external pulse generator 106 (EPG) (not shown). The inside of each lead adaptor 104 can include a series of receptacles which accept a lead connector on the lead 102 to establish electrical connectivity with the EPG 106. The lead 102 is held in place using a retention system, which may include a clamping system and friction channel to secure the lead 102 to the lead adaptor 104. The lead adaptor case can include a communication cable, e.g., Ethernet or other type cable, that connects the lead adaptor 104 to the EPG 106.

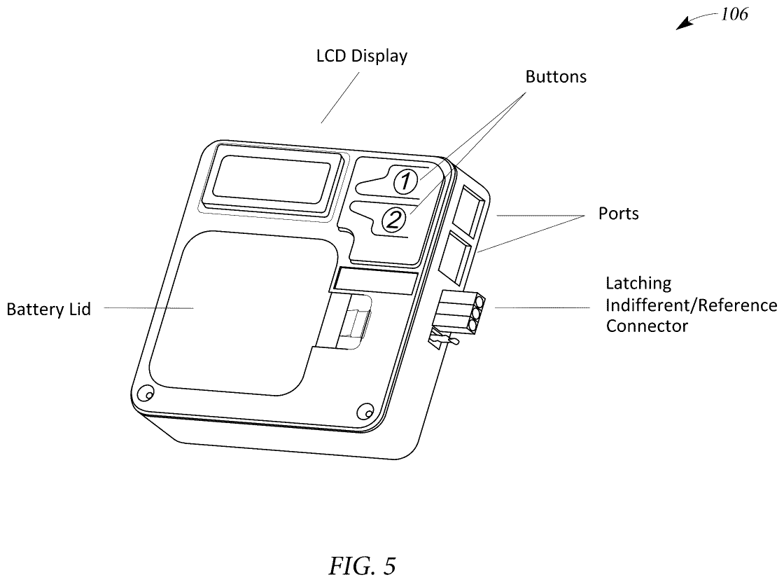

FIG. 5 schematically illustrates an external pulse generator 106 (EPG), according to some embodiments. The EPG 106 is configured to generate electrical, therapeutic pulses, including any of the waveforms described herein. It generally includes an enclosure, such as a box, made of nylon or other appropriate material, as one non-limiting example. The percutaneous lead adaptor 104 (not shown) interfaces with the EPG 106 through connectors or ports, such as two Ethernet-style (e.g., RJ-45, etc.) or other connectors. The EPG 106 power supply can be provided by, for example, four standard primary cell lithium AA batteries. Users can interface with the EPG 106 via, for example, two buttons positioned next to an optional display, e.g., a LCD or other screen. The optional LCD displays the status of the device and battery status to the user. The user is given audible and/or visual alerts to prompt action for states which require the user's attention, such as battery changes, etc. Other visual indicators, such as, for example, LEDs visible through the EPG case or openings in the case provide additional indications to the user. The EPG 106 is also connected to a skin surface indifferent electrode. The indifferent electrode is connected through connector on the EPG 106 such as a latching connector. In some embodiments, an automated external defibrillator electrode is used as the indifferent electrode for well-established safety and low electrical impedance. Additionally, an optional external reference electrode can be available on the same harness. The reference electrode itself can be, for example, is a commercially available electrocardiogram electrode. The EPG 106 can be programmable through a wireless connection to a Clinician Programmer Device (CPD) (not shown) that is USB-connected (or wirelessly connected, etc.) to a PC running a clinician programming application (CPA). Alternatively, the EPG 106 may be directly connected to a PC via a direct USB or other cable (or wireless) connection. The EPG 106 can normally reside, for example, in a belt-mounted holster type pouch that the patient may wear during use. In some embodiments, the system (including but not limited to the external pulse generator 106) could be fully or partially implantable, and include, or not include any transcutaneous components, and/or be configured be controlled or interrogated with an external device.

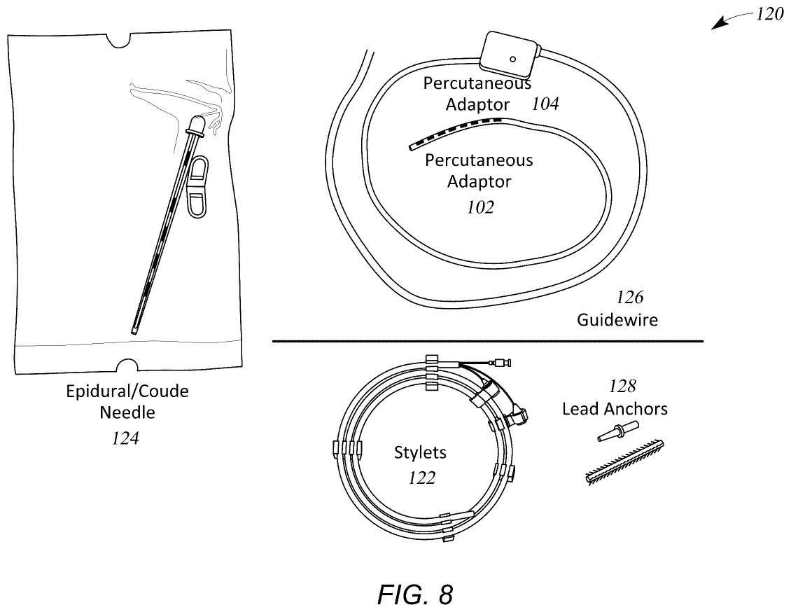

FIG. 6 schematically illustrates an example of an EPG Output Waveform. The EPG output waveform may be referred to as a therapeutic pulse, an EPG output pulse, a therapeutic waveform, a DC waveform, a low frequency waveform, or an ultra-low frequency (ULF) waveform, as used throughout this disclosure and as discussed, for example, with reference to FIGS. 8B, 11, 12, 13, 15A, 15B, 16, 19, 21A, 22A-E, 24, and 25. The EPG outputs pulses which are ramped both up and down. The pulse width, frequency and amplitude can be programmed using the Clinician Programming Device. In some embodiments the EPG 106 or other pulse generator can output a current of between about 0-10 mA, or other values/ranges as disclosed herein (and is configurable to not exceed the electrode charge injection capacity). In some embodiments, the EPG 106 or other pulse generator has an output frequency of between about 0.05 Hz and about 2 Hz, or other values/ranges as disclosed herein. In some embodiments, the EPG 106 waveform 500 has a pulse width of between about 250 ms and about 30 seconds, or other values/ranges as disclosed herein. The EPG waveform 500 described herein may be generated and output by the EPG 106 or other pulse generator system or systems such as a trial pulse generator or implantable pulse generator.

Systems and methods can also include a Clinician Programming Application (CPA)/Clinician Programming Device (CPD). In some embodiments, a computing device with the Clinician Programming Application (CPA) communicates wirelessly with the External Pulse Generator or other pulse generator such as a trial or implantable pulse generator via the Clinician Programming Device (CPD) which is attached via, e.g., USB to the computing device or alternatively through a wireless communication protocol such as Bluetooth. The system uploads the waveform to the EPG or other pulse generator based on the parameters set by the programmer/clinician. It also can have the capability to download logs and performance data from previous stimulation sessions.

One embodiment of such a therapeutic waveform is illustrated in FIG. 6. The waveform 500 includes an anodic (e.g., positive) phase 502 and a cathodic (e.g., negative) phase 504. The waveform 500 also includes an anodic plateau phase 506 and a cathodic plateau phase 508. Transition regions (sometimes referred to as regions where breakthrough conduction occurs) 510, 512 separate the waveform's cathodic and anodic plateau phases 504, 502.

Each transition region 510, 512 can include one or more transition waveform portions. In the illustrated embodiment, the waveform 500 transitions from its cathodic plateau phase 508 to its anodic plateau phase 506 at a first transition region 510. The first transition region 510 includes four transition waveform portions: a progressively increasing curved slope portion 518, a substantially linear slope first portion 520, a substantially linear slope second portion 522, and a progressively decreasing slope curved portion 524. During the progressively increasing curved portion 518, current amplitude is gradually decreased at a slowly increasing rate from the cathodic plateau phase 508 to the first substantially linear portion 520. During the first substantially linear portion 520, current amplitude is decreased at a first substantially constant rate until a transition point 514 is reached. At the transition point 514, current amplitude is increased at a second substantially constant rate until the progressively decreasing curved portion 524 is reached. During the progressively decreasing curved portion 524, current delivery amplitude continues to increase, but at a gradually decreasing rate until the anodic plateau phase 506 is reached.

At the end of the anodic plateau phase 506, the waveform 500 transitions to its cathodic plateau phase 508 at a second transition region 512. The second transition region 512 includes four transition waveform portions: a progressively decreasing slope curved portion 526, a substantially linear first portion 528, a substantially linear second portion 530, and a progressively increasing slope curved portion 532. During the progressively decreasing curved portion 526, current amplitude is gradually decreased at a slowly increasing rate from the anodic plateau phase 506 to the first substantially linear portion 528. During the first substantially linear portion 528, current amplitude is decreased at a first substantially constant rate until a transition point 516 is reached. At the transition point 516, current amplitude is increased at a second substantially constant rate until the progressively increasing slope curved portion 532 is reached. During the progressively increasing slope curved portion 532, current delivery amplitude continues to increase, but at a gradually decreasing rate until the cathodic plateau phase 508 is reached. The cycle is repeated for subsequent waveforms 500.

In one embodiment, a positive current is delivered during the anodic phase, a negative current is delivered during the cathodic phase, and the current reaches zero and transitions from negative to positive, or from positive to negative, at the transition points 514, 516. In some embodiments, the amplitude of the waveform 500 anodic plateau phase 506 is the same magnitude but opposite sign of the waveform 500 cathodic plateau phase 508. In some embodiments, the amplitude of the waveform 500 anodic plateau phase 506 is unequal in magnitude and opposite in sign of the waveform 500 cathodic plateau phase 508. In some embodiments, the plateau period 506 and 508 can be substantially flat with a superimposed AC signal, e.g., a conventional SCS waveform, HFAC waveform, or other alternating current signal. In some embodiments, the waveform 500 is offset or biased by a bias current amount in either the positive or negative direction. In some embodiments, the current reaches the bias current amount and transitions from negative to positive, or from positive to negative, at the transition points 514, 516. Example bias current amounts are discussed throughout this disclosure and may be used in connection with the waveform 500 of FIG. 5. In some embodiments, a small waveform, e.g., a square wave, may be inserted at at least one or more points, e.g., the transition points 514 and 516, to allow for assessment of electrode or tissue properties, e.g., serial solution impedance, electrode impedance, etc.

The waveform 500 of FIG. 6 provides significant clinical advantages when providing a therapeutic waveform to a neural tissue to induce a neural block such that neural activation (e.g., pain sensations, paresthesia, etc.) do not occur. Such advantages are discussed in greater detail herein.

For example, the length or duration of the waveform's anodic and cathodic plateau phases 506, 508 appears as a DC or direct current to neurons. The long, DC-looking plateau has a much longer duration than the typical millisecond time constant of a neuron. The waveform plateau regions 506, 508 can cause an immediate or almost immediate clinical effect of blocking neural activity.

However, during the transition from anodic to cathodic, or from cathodic to anodic plateaus, there is a chance to activate the target neural tissue. To avoid such activation, relatively linear transition portions 520, 522, 528, 530 of increasing or decreasing current are provided to control the rate of current amplitude change experienced by the target neural tissue, and to hold the current amplitude change rate at a constant or substantially constant amount. By providing the current in a linear ramp (either increasing or decreasing), neural activation may be avoided during waveform 500 transition between anodic and periodic phases 506, 508 and between cathodic and anodic phases 508, 506.

In some embodiments, the slope of the first linear transition portion 520 (the first substantially constant rate) is about 4×, 3×, 2×, 1×, 0.75×, 0.5×,0.25×, 0.125×, 0.062× the slope of the second linear transition portion 522 (the second substantially constant rate). In some embodiments, the slope of the first linear transition portion 528 (the first substantially constant rate) is about 4×, 3×, 2×, 1×. 0.75×, 0.5×, 0.25×, 0.125×, 0.062× the slope of the second linear transition portion 530 (the second substantially constant rate).

In addition, the slope of the rising and falling waveforms (e.g., during the increasing transitional phase 510 and during the decreasing transitional phase 512), as well as the maximum amplitude (both anodic and cathodic), individually or together, can affect whether neural activation can be avoided. Therefore, in some embodiments, the slopes of the waveform portions 520, 522, 528, 530, the plateau phase 506, 508 current levels, or both, are selected (e.g., by a processor, microcontroller, EPG, programmer, etc.) to cause neural block or avoid inadvertent neural activation to occur.

Finally, the rounded corners 518, 524, 526, 532 of the waveform 500 provide progressively decreasing rates of current amplitude increases as the waveform 500 transitions into its plateau phases 506, 508 (at regions 524, 532) as well as a progressively increasing rates of current amplitude decreases as the waveform 500 transitions out of its plateau phases 506, 508 (at regions 518, 526). Such rounded corners 518, 524, 526, 532 further prevent inadvertent neural activation as the waveform 500 transitions between anodic and cathodic periodic phases 502, 504.

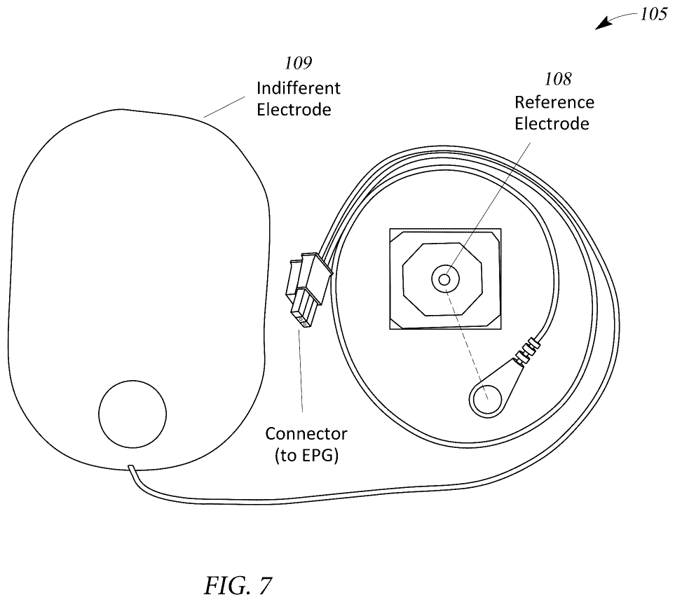

FIG. 7 schematically illustrates a Surface Electrode Harness 105, which can comprise an Indifferent Electrode 109 and a Reference Electrode 108, as well as a connector that interfaces with the External Pulse Generator. The electrodes 108, 109 can serve to enable delivery of the output waveform 500, specifically absorbing the bias current regardless of polarity, and recording of performance data. The electrodes can adhere to the skin and can be further secured with adhesive films such as, for example, Tegaderm as necessary.

FIG. 8 schematically illustrates one embodiment of a sterile lead implantation kit 120, which includes leads and lead adaptors, as well as any number of the following: leads 102 (1 shown); Percutaneous Adaptors 104 (1 shown); (3) Stylets 122 packaged in a round Teflon holder, one straight and two curved-tip stylets 122 of increasing stiffness; (2) Coude needles 124; (1) Epidural access guidewire 126; and (2) Lead retention anchors 128. Not shown is a tunneling tool for full lead implantation.

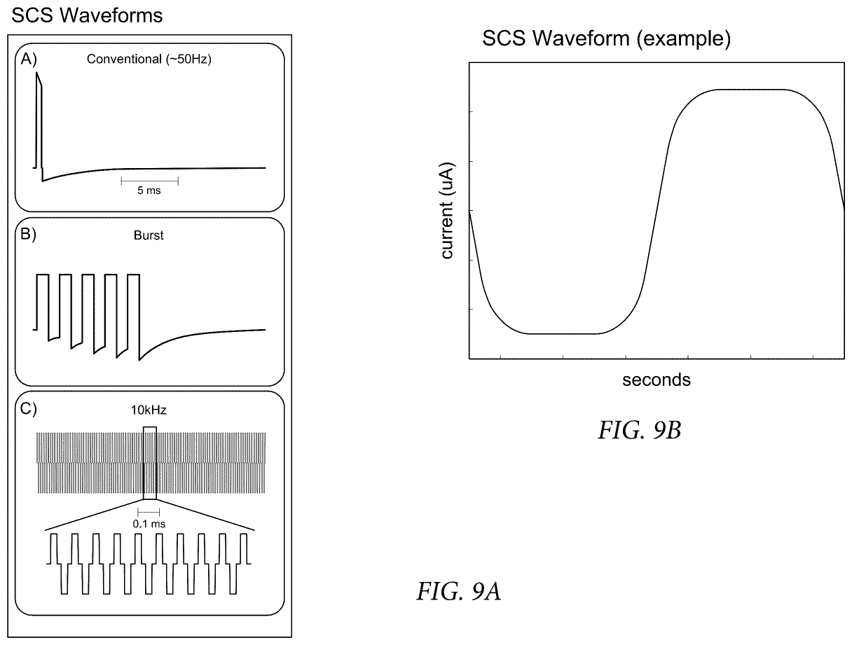

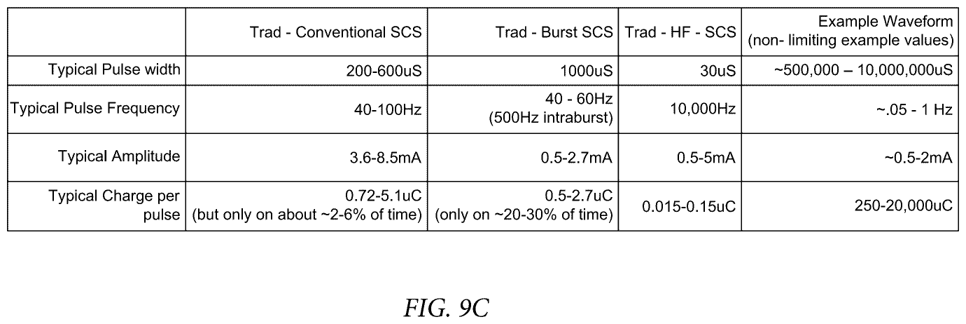

FIG. 9A illustrates example spinal cord simulation (SCS) waveforms, including conventional, burst, and high frequency (e.g., greater than about 1 kHz, or more). As illustrated, each of the foregoing waveforms include sharp angles or corners. In contrast, the SCS waveform illustrated in FIG. 9B (as well as the waveform 500 of FIG. 6) includes curves in place of sharp angles, corners, or edges, which can provide any number of the benefits explained herein. The SCS waveform illustrated in FIG. 9B (as well as the waveform 500 of FIG. 6) can be referred to as a spline waveform shape and/or smooth waveform shape instead of the square wave or sharp corner shapes illustrated in FIG. 9A. FIG. 9C illustrates a table with example non-limiting pulse widths, pulse frequencies, amplitudes, and charge per pulse values for each of the conventional, burst, high frequency, and curved SCS waveforms. As provided in the table, the charge per pulse value for the curved SCS waveform illustrated in FIG. 9B can be much greater than, and even orders of magnitude greater than that of the other SCS waveforms.

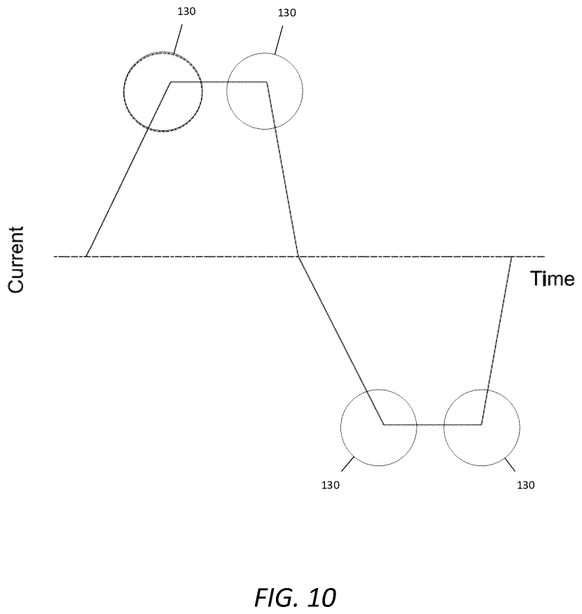

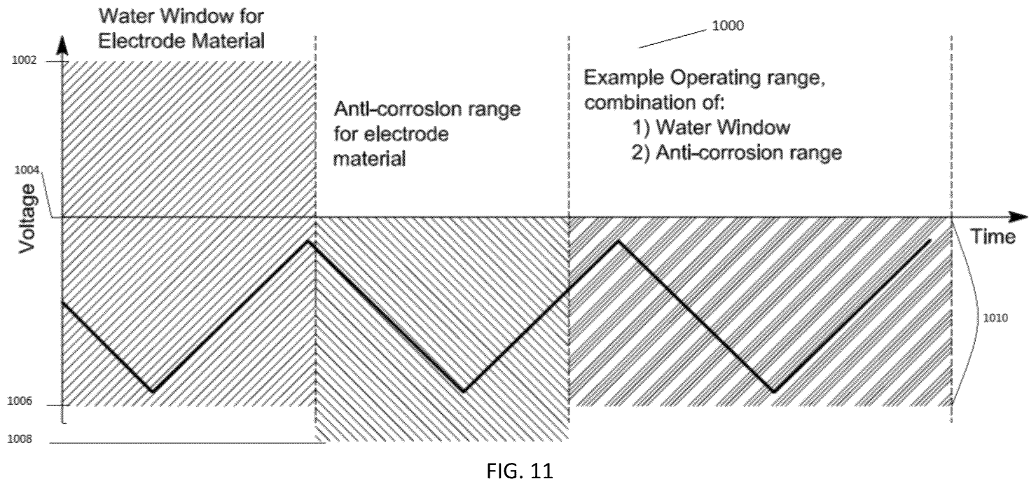

FIG. 10 illustrates a SCS waveform with sharp corners or angles. In some embodiments, sharp edges on stimulation waveforms can cause undesired responses. For example, the circled regions 130 indicate where undesirable motor activity (hind-leg contractions in animal models) have been observed. Rounding the sharp angles, corners, or edges can advantageously increase the amplitude that can be driven prior to generating motor activity.

FIG. 11 illustrates an example graph 1000 representative of different electrode operating ranges. In one embodiment, an electrode operates in a favorable operating voltage range 1010 to limit potentially irreversible byproduct formation (e.g., chemical byproducts) and to promote electrode longevity. Promoting electrode longevity can include protecting the electrode from undesirable reactions (e.g., corrosion, etc.). The graph shows voltage over time with a water window for electrode material, anti-corrosion range for electrode material, and an example operating range for a combination of water window and anti-corrosion ranges.

A water window range defines an operating range for an electrode within which undesirable electrode reactions (e.g., due to water electrolysis, etc.) will occur to a minimal extent. The water window range has an upper voltage limit 1002 and a lower voltage limit 1006. For example, in one embodiment, the water window range has an upper voltage limit 1002 and a lower voltage limit 1006 of 0.8 V and −0.6 V, 1.4 V and −1.4 V, 0 V and −0.6 V, 0 V and −1.4 V, 0.2 V and −0.6 V, 0.2 V and −1.4 V. As long as the electrode is operated within the water window, between the upper and lower voltage limits 1002, 1006, significant breakdown of water via water electrolysis may be avoided. In addition, an anti-corrosion range defines an operating range for an electrode within which undesirable electrode corrosion will not occur. The anti-corrosion range has an upper voltage limit 1004 and a lower voltage limit 1008. As long as the electrode is operated within the anti-corrosion range, between the upper and lower voltage limits 1004, 1008, electrode corrosion may be avoided. In one embodiment, the upper voltage limit 1004 is 0 V, 0.1 V, 0.2 V, or between 0 V and 0.2 V. An operating range 1010 for an electrode is therefore selected to assure that the electrode is always operated within both (e.g., within the overlap of) the water window range and the anti-corrosion range for the particular electrode. This can be generalized that an electrode should be run in a voltage range to limit undesired electrochemical reactions to avoid impact on electrode (e.g., via hydrogen embrittlement, etc.) or biology (e.g., via generation of hydrogen peroxide or other reactive oxygen species). In the illustrated embodiment, the operating range 1010 is defined by the upper limit 1004 of the anti-corrosion range and the lower limit 1006 of the water window. Furthermore, the upper limit 1004 of the anti-corrosion range may be at 0 V, greater than 0 V, or less than 0 V. A bias current (such as discussed above and below) may be selected to control the operating range 1010 of the electrode, and to assure that electrode operation occurs in the desired range, within the water window and anti-corrosion range of the electrode and any other undesired reactions.

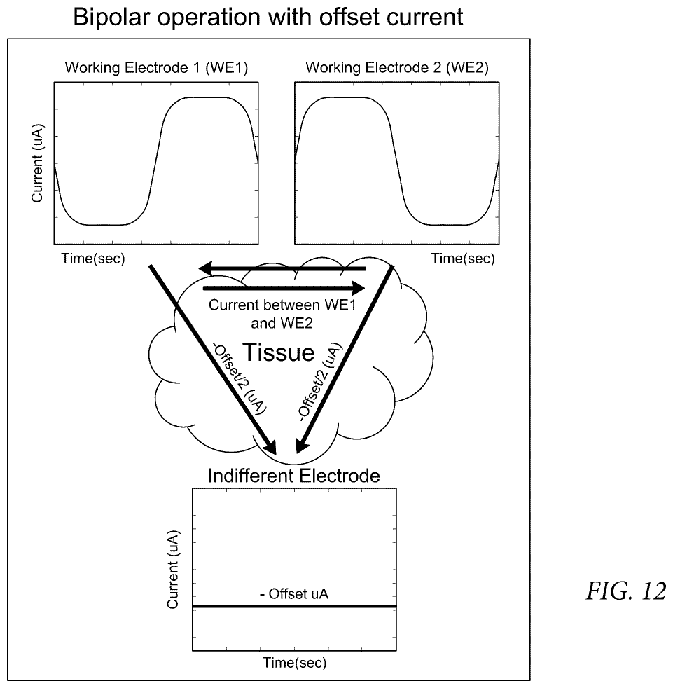

FIG. 12 illustrates an example bipolar operation with bias (offset) current. In a bipolar configuration, balanced low frequency stimulation current is conducted between two working electrodes (WE1, WE2). An offset current is added to each WE electrode and is absorbed by the indifferent electrode, which can be either a surface or implanted transcutaneous electrode that can accommodate the opposite voltage of the working electrode. FIG. 12 shows a non-limiting example of the bipolar arrangement with even bias (offset) currents for each electrode and a constant DC bias (offset) current. In a monopolar arrangement, stimulation current is conducted between the working electrode (WE) and a counter or indifferent electrode (IE). The counter or indifferent electrode can be a surface or implanted electrode or transcutaneous electrode that can accommodate the opposite voltage of the working electrode. Stimulation current can include an ultra low frequency AC component and an offset current can be added such that the WE is biased in the correct voltage range. In another embodiment, the low frequency stimulation current is provided according to a waveform, such as the waveform illustrated in FIG. 6.

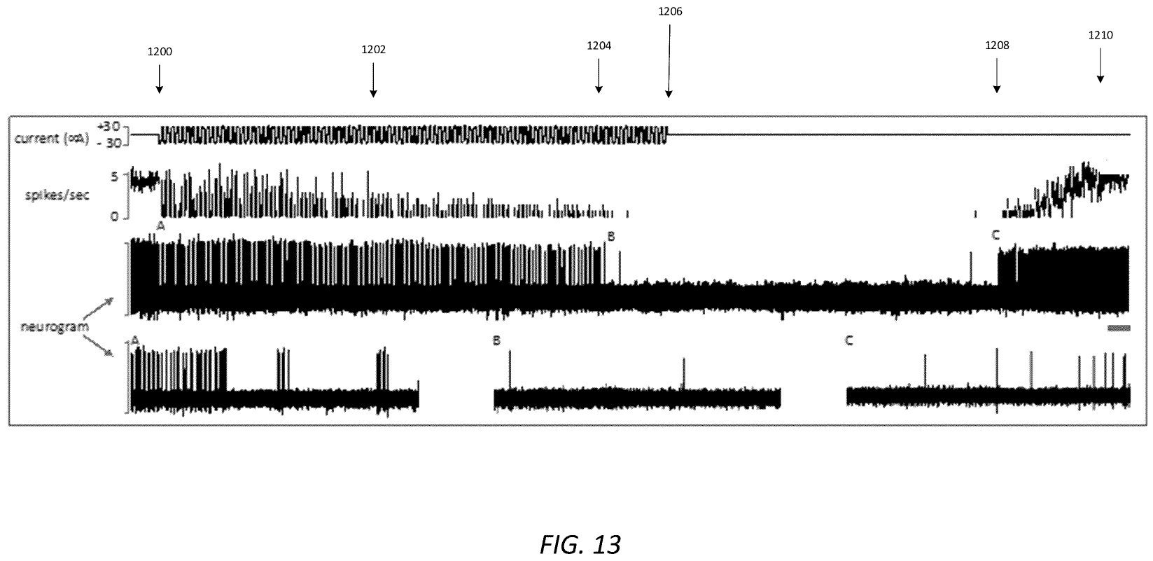

FIGS. 13-15 relate to long lasting neural block from ultra-low frequency currents (ULF) (e.g., using the waveform 500 of FIG. 6), which can include slowly developing block and recovery of conduction of neural activity, such as from ectopic firing in a neuropathic state. The prolonged use of a biphasic ULF waveform can advantageously enable continued suppression of neural activity after cessation of current delivery (wash-out period), which can provide multiple benefits.

FIG. 13 illustrates a recording from a filament of L5 root with ectopic firing in a neuropathic state recorded 8 days after axotomy by section of the L5 spinal nerve. The aggregate discharge rate (e.g., spike rate) for the active fibers is approximately 5 spikes/second at baseline. When ULF current (e.g., 30 μA or any other current value discussed herein) is applied to the dorsal root at time point 1200 (e.g., applying the waveform 500 of FIG. 6), distal to the recording site, there is a block of conduction at both cathodal and anodal plateau phases with breakthrough conduction at phase crossover. After six minutes at time point 1202, only one of the active units maintains breakthrough conduction and after 20 minutes at time point 1204, full conduction block is achieved in both fibers. The current is then switched off at time point 1206, yet full conduction block continues during a recovery phase between time points 1206 and 1208. Recovery of conduction in one of the fibers begins 12 minutes later at time point 1208. Full restoration of conduction in both fibers is seen at 17 minutes after the current is discontinued at time point 1210. Scale bar, 1 minute. Below the main neurogram, A-C are excerpts, each of 20 second duration, from the continuous recording, showing detail of spike activity at points indicated.

Interestingly, a full conduction block is maintained in the neural tissue for a full 12 minutes after the ULF current waveform is removed from the tissue (e.g., turned off). In addition, full neural activity resumes only five minutes after the neural tissue begins to show recovery (from time point 1208 to time point 1210). The ULF waveform is therefore able to almost immediately cause a partial neural block (during anodal and cathodal plateau phases) as soon as it is applied to neural tissue (e.g., at time point 1200), with a full neural block following only a few minutes after the ULF waveform is applied (e.g., at time point 1204). At lower ULF waveform amplitudes, it has been demonstrated that a partial block can be achieved with a delay from the time of application of the ULF waveform (e.g. a non-immediate partial neural block). Furthermore, a secondary blocking effect occurs once the ULF waveform is removed (e.g., at time point 1206) and continues for a substantial period (e.g., until time point 1208). Full neural activity resumes a short time later (e.g., at time point 1210).

Advantageously, such therapeutic waveform can provide a pain therapeutic that does not simultaneously create an undesirable anesthesia, paresthesia, or numbness that is sensed by the patient. Furthermore, power efficiency may be improved by utilizing controlling or cycling current delivery (e.g., via ULF or therapeutic waveform 500 of FIG. 6, etc.), which may be deactivated during the neural recovery period (e.g., from time point 1206 to time point 1208). Furthermore, less current may be provided to achieve neural block by taking advantage of this secondary blocking effect (e.g., continued neural activity blocking post-waveform removal), which means that tissues surrounding the target tissue will be less affected or completely unaffected by the therapeutic waveform and any potential by products generated by the waveform.

In another study it was found that the application of the ULF waveform results in a reduced “windup” response. A windup response refers to an increased sensitivity, or hypersensitivity, of a neuron that has experienced chronic, long-term pain stimuli. The ULF waveform can selectively reduce the pain or other neural response from travelling to the brain. For example, in many situations it is not desired to completely block the neural pathway; instead, it is advantageous to merely reduce the windup response, resulting in maintaining an acute pain response, but reducing or eliminating the neural hypersensitivity associated with windup and chronic pain. Furthermore, neural hypersensitivity reduction (or removal) may continue even after removal or cessation of the ULF waveform occurs.

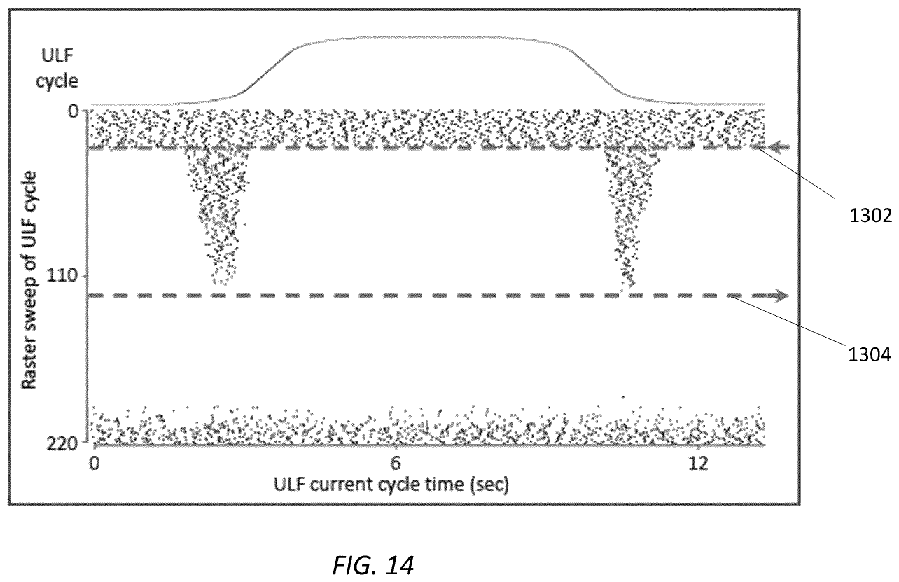

FIG. 14 illustrates a raster plot showing the block and recovery of conduction in the spontaneously active fibers in FIG. 13. FIG. 14 illustrates another method of visualizing the neurogram data of FIG. 13. At the top of the plot, dots indicate baseline firing of the units. As the waveform current is maintained, the window during which conduction is possible narrows until full block is achieved. For example, the width of the horizontal dotted portions of each dot row narrows from the upper dashed horizontal line to the lower dashed horizontal line. The dashed horizontal lines 1302, 1304 indicate ULF current on (the upper horizontal dashed line 1302) (corresponding to time point 1200 of FIG. 12) and off (the lower horizontal dashed line 1304) (corresponding to time point 1206 of FIG. 12). The time period between the upper horizontal dashed line 1302 and the lower horizontal line 1304 (corresponding to the time period between time point 1200 and time point 1206 of FIG. 12) may be referred to as the ULF application period. Each horizontal row of dots (each row being one dot thick), represents one period of the therapeutic waveform (e.g., the waveform 500 of FIG. 6 or 13). Each dot represents an action potential, spike, or activation of the neuron. In some embodiments, it may be desirable to utilize waveform properties, e.g., current levels, such that breakthrough conduction is preserved throughout the duration of the therapy, such as the activity shown somewhere between the upper and lower horizontal dashed lines 1302, 1304. In this manner, the transmission of neural signals may be modulated from high to low values or eliminated completely.

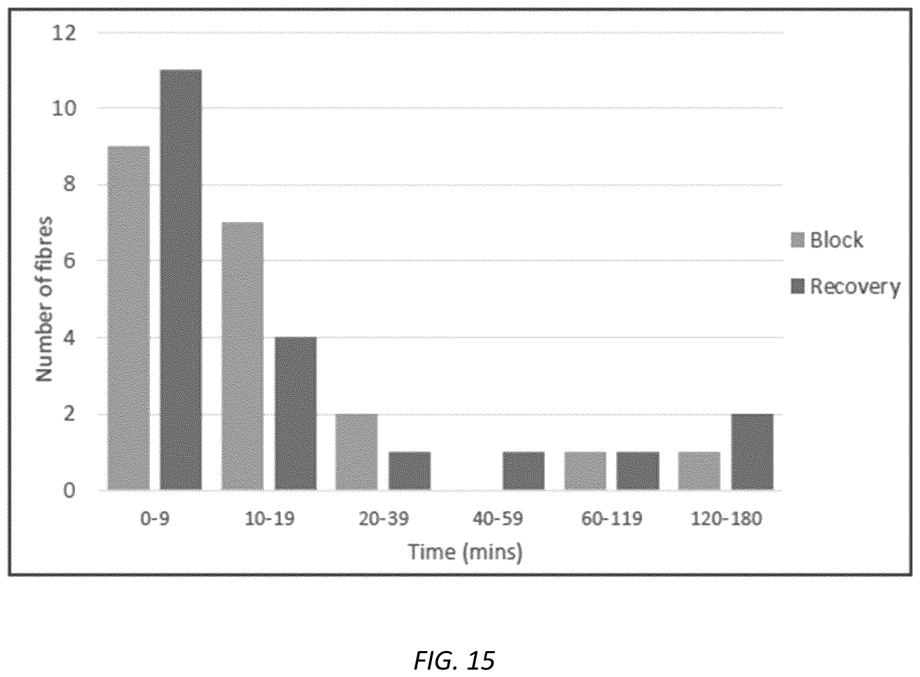

FIG. 15 illustrates a distribution plot showing block and recovery times in 20 tests on a sample of 11 fibers where full conduction block was achieved during ULF current. The distribution plot illustrates the variability in how long it takes fibers to be blocked or to recover. Such variability may be due to the distance between the electrode and the respective nerve fiber.

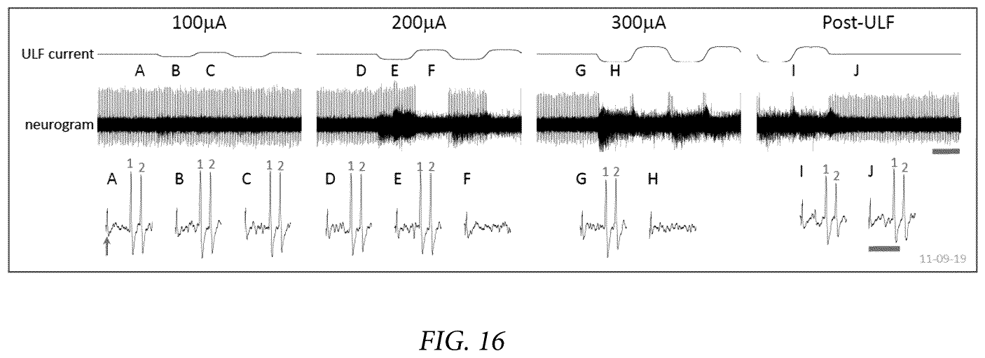

FIG. 16 relates to the ability to deliver a partial block (e.g., less than a complete block), which can include acute block of conduction in intact fibers. The partial block of neural activity can be produced using a ULF waveform. In one example, relative phase of time of plateau versus breakthrough phase can be used to generate a partial block over the waveform period. Conduction is blocked during plateaus for some time t1, while conduction is allowed during transitions for some time t2. The ratio of t1:t2 can then control the amount of block generated during the waveform period. For example, a partial block can be achieved by providing a block during certain (or all) plateau phases (where the waveform is horizontal) and by allowing neural signal transmissions during transition periods between the plateau phases. In one embodiment, if the waveform's plateau time in one period is t1 and its transition time is t2, then a partial block may be defined as a function of t1 and t2, such as a function of the ratio of t1 to t2, or of t2 to t1. If t1>t2, then a partial block of >50% may be achieved. If t2>t1, then a partial block of <50% may be achieved. In another example, cathodic/anodic threshold-blocking and pass through may have different thresholds on anodic verses cathodic, which can be utilized to permit and block during associated phases. Partial block can enable the tunable block of neural activity for systems where a complete block is not desirable. For example, it may not be desirable to have a complete block of signaling for mobility disorders to allow for mobility during therapy application. Additionally, a complete block of signaling to avoid side-effects (e.g., gastric distress from sympathetic block) may not be desired with sympathetic modulation. Further, it may be desirable to block neural activity such that cessation of the block results in immediate recovery of the nerve, such as that developed prior to complete block of the breakthrough conduction.

FIG. 16 illustrates recordings from a filament containing two A fibers (conduction velocities approximately 19 meters/second and 14 meters/second) activated by electrical stimulation of the ipsilateral hind paw at 5 Hz. At a current of 100 μA, there is no block of conduction. At 200 HA, conduction block occurs at the anodal plateau phase of the current cycle and this current is defined as the threshold (T) for conduction block. Increasing the current to 300 μA produces conduction block at both anodal and cathodal phases. Therefore, a partial neural block may be realized by controlling the amplitude of the therapeutic waveform (e.g., waveform 500 of FIG. 5 or FIG. 12) and setting the waveform amplitude to a desired level. Furthermore, based on timing or superposition of multiple waveforms, it is possible to tune the types of neural fibers that are blocked, for example, based on their conduction velocity or fiber time. Stimulation levels may be titrated to implement selective neural block control. Furthermore, waveform amplitude may be controlled to compensate for the level of wash-in (e.g., neural hypersensitivity) that may be occurring for a particular patient, as well. In some embodiments, an electrode is used to sense neural activation, and to provide feedback that is used for amplitude level control. For example, as the neural activity is decreased, the waveform amplitude may be decreased (or turned off) in response thereto. Similarly, as neural activity begins to recover, waveform amplitude may be increased (or turned on) to control (or block) neural response. In one embodiment, there is a linear, exponential, or logarithmic relationship between waveform current amplitude and percentage of neural block.

Furthermore, when controlling neural block by controlling waveform current amplitude, no or limited wash-out effect occurs. For example, at point J in FIG. 16, the nerve quickly returns to its original state as soon as (or shortly after) the ULF waveform is removed. The recovery period is either zero or very small, compared to the wash-out effect that occurs during ULF therapy as illustrated in FIG. 13. Limiting or preventing wash-out effect may also be possible by controlling waveform period and/or the total charge delivered in each phase.

Breakthrough conduction is noted at the crossover between cathodal and anodal current phases. When this current is switched off after 4 minutes, normal conduction resumes immediately. Scale bar, 5 seconds. A-J are excerpt traces showing detail of spikes (average of 4-8 sweeps; except B, single sweep; scale bar 5 ms).

Referring back to FIG. 13, the illustrated graph illustrates a return (which may be gentle) to neuronal activity, which can include recovery of neural signaling after cessation of therapy is gradual over time (e.g., the course of minutes to hours). There are various benefits in a gentle return to neuronal activity. For example, the sudden return of neuronal activity can be prevented, which can improve safety. The sudden onset of pain (or other neural activity) after cessation of therapy can be prevented. The sudden onset of sympathetic signaling after cessation of therapy that may lead to undesirable acute events can be prevented, such as acute decomposition or cardiac arrhythmias. In another example, duty cycling with unperceived lack of therapy absence can be achieved. A gradual increase in neural signaling can be used to trigger the delivery of more therapy, ensuring that physiologic markers or symptoms do not return prior to onset of new therapy (e.g., pain can always or substantially always be kept below perceived threshold).

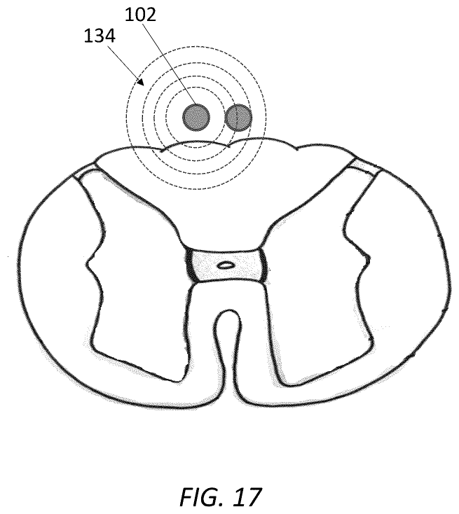

Functional selectivity can advantageously be achieved. Fibers that are close to the electrode can be selectively inhibited, which can leave distal fibers unaffected. Increasing current can increase blocking of distal fibers. Functional selectivity can facilitate the ability to modulate neural activity based on proximity to the electrode and current amplitude, which can prevent off-target effects. FIG. 17 illustrates a cross-section of the spinal cord with leads 102 placed dorsally. As illustrated, a lead 102 is dorsally placed with field lines 134 radiating out from the lead 102 hitting the outermost lamina first. Increasing current can increase field strength at a given radius. The field of the delivered waveform (e.g., waveform 500 of FIG. 6 or 13) can affect the axon, cell body, dendrites of the cell body, or terminal (or end portions) of the axon as well as glial cells such as astrocytes, pericytes, microglia, and oligodendrocytes. Various fibers extend to different laminas of the spinal cord. C fibers (which sense pain) are generally located in the dorsal lamina, closest to the implanted electrode.

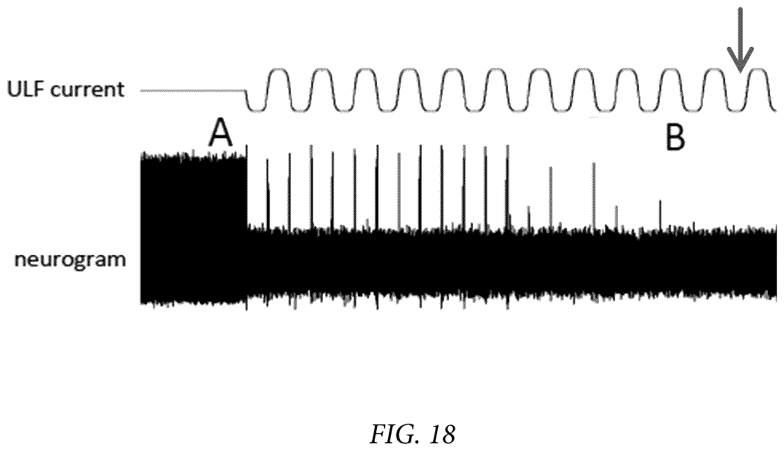

FIG. 18 relates to biphasic ULF waveform for complete block, which can include slowly developing block and recovery of conduction with evoked firing in normal neurons. As discussed in further detail above, breakthrough conduction, or the conduction during a polarity change of a ULF waveform (e.g., waveform 500 of FIG. 6 or 13) occurs during a first period (between points A and B), but stops occurring after such period (at point B). Complete block can be achieved with a biphasic ULF waveform where cathodic current followed by anodic current results in block reversal and recovered signaling. There can be numerous benefits to complete blocking via a biphasic ULF waveform, which can include material safety/feasibility benefits such as the ability to safely establish complete block therapy using material suitable for medical device integration, wherein the biphasic waveform prevents accumulation of irreversible, damaging byproducts. In some embodiments, a single monopolar electrode suitable for medical device integration (opposed to requiring multiple electrodes) can be used to achieve complete block by providing the waveforms described herein. For example, in some embodiments, complete block may be achieved by providing a waveform described herein and causing the nerve to be placed in a state of hypersuppression. In another embodiment, complete block may be achieved by providing a waveform described herein and blocking neural activity during the anodic phase. This may result from creating a virtual cathode proximal and distal to the electrode that results in a cathodic current flow relative to the nerve.

In another embodiment, current modulation for rapid block onset and low power maintenance therapy can include developing block and recovery of conduction in neural activity such as ectopic firing in a neuropathic state. Complete block can be a function of current amplitude and duration of application. A complete block may be quickly achieved by first utilizing high current amplitudes and then reducing current to a lower level for steady-state block maintenance. For example, during a high current amplitude mode, a current having a first amplitude of 800-3000 μA or at least 800 μA is applied, and during a lower level mode a current having a second amplitude that is less than 800 μA is applied. The first and second amplitudes may be expressed in terms of a perception threshold, as well. For example, the first amplitude may be at least 50% of a perception threshold, and the second amplitude may be 25%-50% of, or less than 50% of the perception threshold. The perception threshold may be 25, 50, 100, 200, 400, 600, 800, 1000, 1200, 1400, 1500, 1600, 1800, 2000, 2200, 2400, 2600, 2800, or 3000 μA. Current modulation for rapid block onset and low power maintenance therapy can provide various benefits. For example, reduced power consumption over the long-term can be achieved by consuming high-power only during the initial block onset. The maintenance current may be less than that needed for generating the initial block with low current. In another example related to block speed, a conduction block can be quickly set up to treat acute events. In an example related to electrode safety, the electrode lifetime can be prolonged via reduced charge delivery through the electrode with maintenance current.

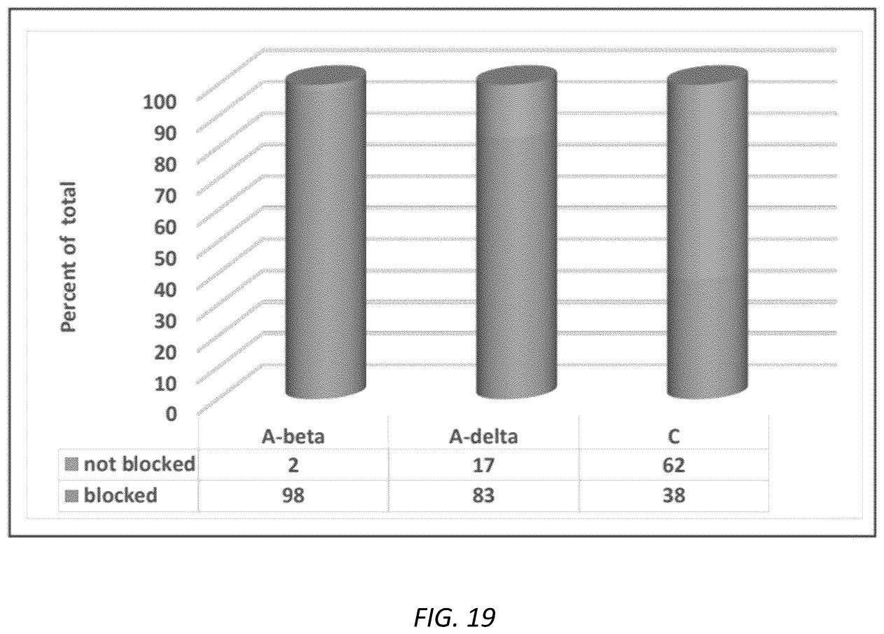

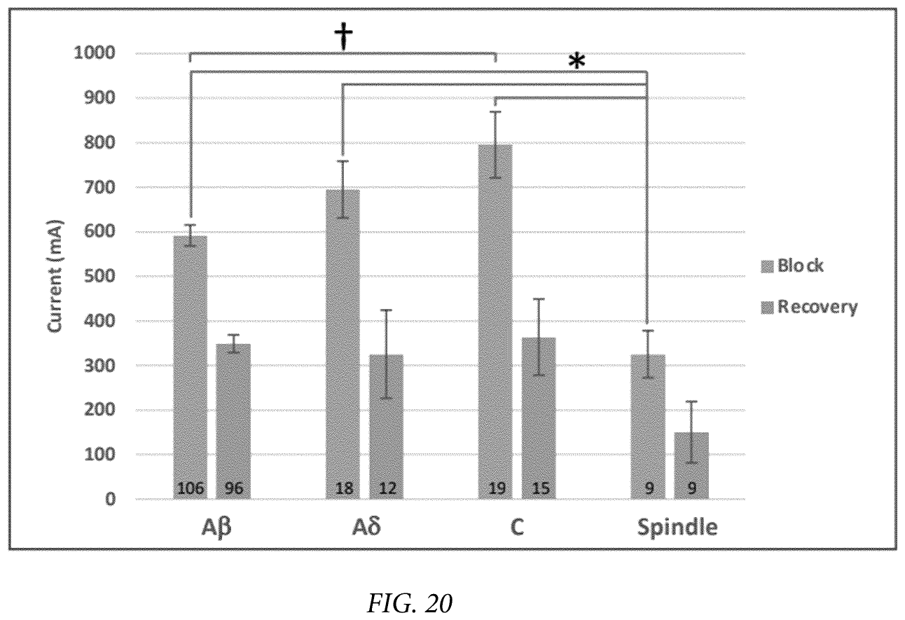

FIG. 19 illustrates a histogram showing the percentages of different fiber types in which conduction could be blocked at currents up to 2000 μA or more or less. FIG. 20 illustrates thresholds for block and recovery from current application in different fiber types including A-beta, A-delta, and C fibers (data from anodal and cathodal staircase currents combined; bars labelled with n values; unequal n for block and recover reflect failure to recover in a small number of units within observed window). One-way ANOVA, Turkey's post-hoc multiple comparisons test, † Aß fibers v. C fibers, P=0.0043; *spindle units vs. all other groups, P=0.0083-<0.0001.

Selections can be based on various parameters, which can include at least conduction velocity, fiber, and/or disease state type. For example, ULF applied at certain currents can provide conduction through the “breakthrough” phase, which can provide various benefits. For instance, the spacing and relative timing of ULF stimulation at electrodes can provide selectivity conduction velocity (which can be a function of fiber type) if several electrodes are spaced along the length of an axon or neural circuit. The fiber type selectivity can allow selective block/pass of certain fiber types and can allow for tailoring of ULF therapy to block/pass certain type activity, which can limit unwanted side effects. In another example, ULF may affect axons/neurons in the disease state differently than naïve, which can provide various benefits. DC current, for example, can have preferential effect on certain channels/gates that may only arise in disease/pain models. For instance, silicon modeling has indicated that ULF affects neurons based on changes on intracellular/extracellular ion concentrations, which can affect different gates differently such that ULF can have an effect on specific disease states gates, axons, and/or neurons.

Modulation of selective weights of network can be implemented. ULF can affect axons, cell bodies, synapses pre and post terminal, and/or dendrites, which can provide various benefits. For example, ULF can affect neurons or synapses, which can change the selective weights of the network which may not be like “axonal block” but effectively alter the network to treat a disease state.

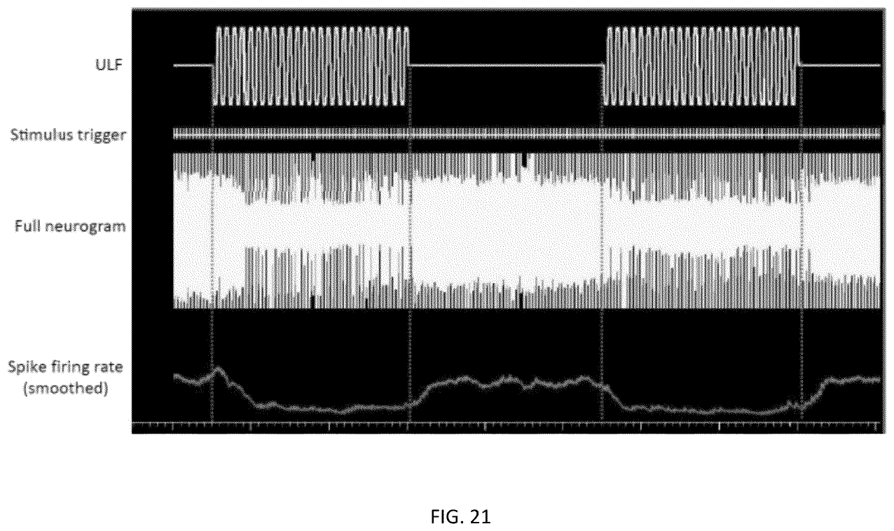

Wide dynamic range (WDR) neurons in the spinal cord can be affected by a ULF waveform, as illustrated in FIG. 21. FIG. 21 shows a recording of the block of foot shock-evoked responses in a dorsal horn neuron by a ULF waveform applied to a cord surface. The WDR neuron 400 μm below the cord surface (lamina V); ULF electrode approximately 1 millimeter rostral to recording site. C,D. Inhibition at both negative and positive 2000 μA spline (2×5 minutes) followed by rapid full recovery. As soon as the ULF waveform is applied, the neural firing rate decreases quickly. As soon as the ULF waveform is removed (turned off), the neural firing rate increases quickly. FIG. 21 illustrates a nociceptive pain response illustrating this response.

Nerve conduction was evaluated in a neuropathic model to investigate the neuropathic disease conditions commonly associated with chronic pain. Nerve ligature was used to generate ectopic firing of the nerve. The experimental model allowed for the application of ULF and step-wise DC waveforms and measurement of influence on nerve fiber conduction.

ULF currents of up to 2000 μA were applied to strands in which 156 single units were assessed. These fibers were classified on the basis of conduction velocity as Aβ (26.0±0.8 m/sec, mean±SEM; range 10.4-70.4 m/sec; n=109), Aδ (4.9±0.4 m/sec; range 1.6-9.8 m/sec; n=14), or C fibers (0.9±0.1 m/sec; range 0.5-1.5 m/sec; n=25). Additionally, a small number of muscle spindle or joint proprioceptive units with ongoing discharge (n=6) and unclassified spontaneously discharging fibers (n=2) were recorded. ULF current produced axonal conduction block in all classes of afferent fibers, but to a different degree, as described below.

Electrical stimuli were applied to peripheral receptive fields at 0.5 Hz to 10 Hz during the ULF current waveforms. The spikes of two afferent fibers in the same filament were recorded before, during, and after short periods of ULF current at three different intensities. At the lowest current (100 μA), there was no effect on conduction during either phase of the ULF waveform. When the current was increased to 200 μA, conduction block in both fibers was seen first at the anodal current plateau phase and, with a further increase in current to 300 μA, at both anodal and cathodal phases. As the current transitions between negative and positive phases, conduction is briefly restored in both afferents. When ULF currents were switched off after a short duration, conduction was immediately restored in both these afferent fibers. Conduction was often transiently restored as the ULF current transitioned from one polarity to the other. This phenomenon, referred to a break-through conduction, was commonly observed.

ULF at threshold amplitudes can block action potentials immediately during plateau phase. At lower amplitudes, a slower wash-in block can occur. ULF can have a washout effect that can last for a substantial time.

The wash-in/wash-out effect can be mediated by ion accumulation. Delivery of ULF results in transmembrane ion flow (e.g., Na+ and K+). The net ion flow is down the ion concentration gradient across the membrane. At a sufficient amplitude, the degree of ULF-induced ion flow may be greater than the reverse ion flow induced by the transmembrane sodium/potassium pump proteins. With prolonged delivery of ULF, the decrease in ion concentration gradients results in a depolarizing shift in the Nernst potential and a corresponding blockade of action potential conduction (in axons), or inhibition of excitability (cell bodies and axons). In Silico models can recreate major components of this phenomenon. The speed of wash-in effect (e.g. 1's of minutes, 10's of minutes, hours, days) can increased by increasing the amplitude of ULF (e.g., in the range of 0.05 mA-0.1 mA, 0.1 mA-0.5 mA, 0.5 mA-1.0 mA, 1.0 mA-5 mA, 5 mA-10 mA). A lower amplitude ULF signal delivered for a longer period of time can achieve neural inhibition/suppression/blockade using less amplitude/power (for example, at approximately 95%, 90%, 80%, 70%, 60%, 50%, 40%, 30%, 20%, 10%, 5% of the ULF amplitude required for effectiveness without wash-in).

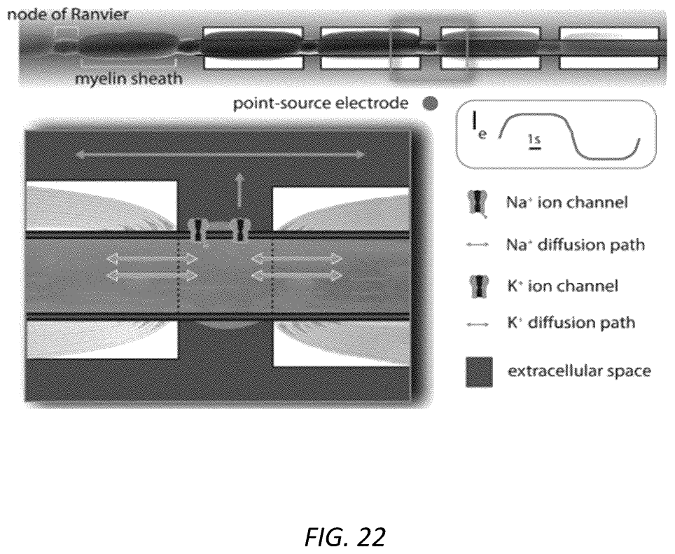

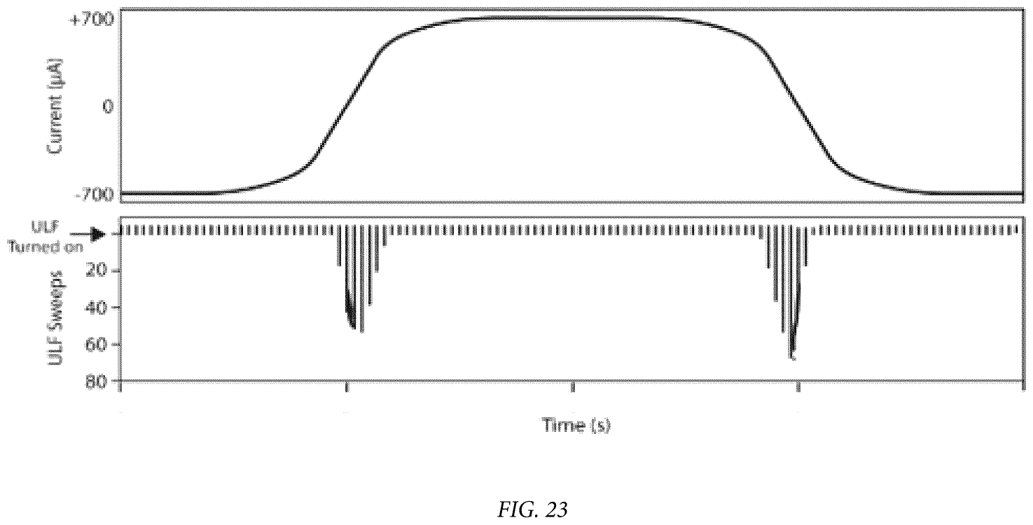

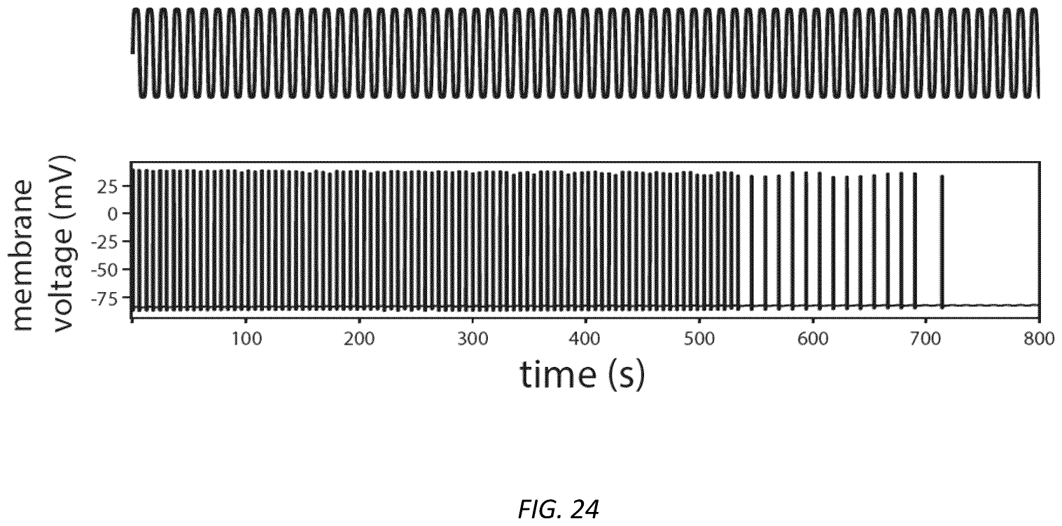

FIG. 22 is a schematic representation of the node of Ranvier with illustrated ion channels and paths. FIG. 23 illustrates a raster sweep. FIG. 24 illustrates a membrane voltage (mV) over time in seconds.

In some embodiments, systems and methods as disclosed herein can be used or modified for use as part of alternating current stimulation systems, including but not limited to spinal cord stimulation (SCS) systems for treatment of chronic pain, such as for example the SENZA® system by Nevro Corporation; the PRECISION® systems including PRECISION PLUS® and PRECISION SPECTRA® by Boston Scientific Corporation, the INTELLIS® system from Medtronic PLC, and the PROCLAIM® system from Abbott. As one example, systems and methods as disclosed herein can increase efficacy of an alternating current delivery system including delivering alternating current via an electrode and electrode lead to a target tissue of a patient utilizing a DC-offset waveform generated by a pulse generator and facilitated by a controller. The alternating frequency could be any desired frequency, including high frequency systems of about 10 kHz or higher, from about 1.5 kHz to about 100 kHz, from about 1.5 kHz to about 50 kHz, from about 3 kHz to about 20 kHz, from about 3 kHz to about 15 kHz, from about 5 kHz to about 15 kHz, or from about 3 kHz to about 10 kHz, or other ranges incorporating any two of the aforementioned values. The electrode and/or electrode lead can include one or more of: high density charge materials, a SINE electrode, and/or a silver-silver chloride material. Such systems and methods can in some cases advantageously increase the excitability of target neurons, thereby decreasing thresholds and widening the therapeutic window of the target tissue stimulation.

The foregoing description and examples has been set forth to illustrate the disclosure according to various embodiments and are not intended as being unduly limiting. The headings provided herein are for organizational purposes only and should not be used to limit embodiments. Each of the disclosed aspects and examples of the present disclosure may be considered individually or in combination with other aspects, examples, and variations of the disclosure. In addition, unless otherwise specified, none of the steps of the methods of the present disclosure are confined to any particular order of performance. References cited herein are incorporated by reference in their entirety.

While the methods and devices described herein may be susceptible to various modifications and alternative forms, specific examples thereof have been shown in the drawings and are herein described in detail. It should be understood, however, that the embodiments disclosed should cover modifications, equivalents, and alternatives falling within the spirit and scope of the various embodiments described herein and the appended claims.

Depending on the embodiment, one or more acts, events, or functions of any of the algorithms, methods, or processes described herein can be performed in a different sequence, can be added, merged, or left out altogether (e.g., not all described acts or events are necessary for the practice of the algorithm). In some examples, acts or events can be performed concurrently, e.g., through multi-threaded processing, interrupt processing, or multiple processors or processor cores or on other parallel architectures, rather than sequentially.

The use of sequential, or time-ordered language, such as “then,” “next,” “after.” “subsequently.” and the like, unless specifically stated otherwise, or otherwise understood within the context as used, is generally intended to facilitate the flow of the text and is not intended to limit the sequence of operations performed.

The various illustrative logical blocks, modules, processes, methods, and algorithms described in connection with the embodiments disclosed herein can be implemented as electronic hardware, computer software, or combinations of both. To clearly illustrate this interchangeability of hardware and software, various illustrative components, blocks, modules, operations, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration.

The blocks, operations, or steps of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, an optical disc (e.g., CD-ROM or DVD), or any other form of volatile or non-volatile computer-readable storage medium known in the art. A storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

Conditional language used herein, such as, among others, “can.” “might,” “may.” “e.g.,” and the like, unless specifically stated otherwise, or otherwise understood within the context as used, is generally intended to convey that some examples include, while other examples do not include, certain features, elements, and/or states. Thus, such conditional language is not generally intended to imply that features, elements, blocks, and/or states are in any way required for one or more examples or that one or more examples necessarily include logic for deciding, with or without author input or prompting, whether these features, elements and/or states are included or are to be performed in any particular embodiment.

The methods disclosed herein may include certain actions taken by a practitioner; however, the methods can also include any third-party instruction of those actions, either expressly or by implication. For example, actions such as “positioning an electrode” include “instructing positioning of an electrode.”

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 hour” includes “1 hour.” Phrases preceded by a term such as “substantially” include the recited phrase and should be interpreted based on the circumstances (e.g., as much as reasonably possible under the circumstances). For example, “substantially perpendicular” includes “perpendicular.” Unless stated otherwise, all measurements are at standard conditions including temperature and pressure. The phrase “at least one of” is intended to require at least one item from the subsequent listing, not one type of each item from each item in the subsequent listing. For example, “at least one of A, B, and C” can include A, B, C, A and B, A and C, B and C, or A, B, and C.

What is claimed is:

1. A system for electrically modulating tissue, comprising:
a current generator;
at least one implantable working electrode, the at least one implantable working electrode configured to be in electrical communication with the current generator;
at least one indifferent electrode; and
a controller configured to signal the current generator to:
generate a set of currents with a set of initial polarities to be delivered to the working electrodes for a time period; and
wherein the magnitudes of the currents are less than a threshold current amplitude corresponding to a current amplitude required to achieve acute blockade in neural tissue, and
cause neural suppression in the neural tissue after the time period.

2. The system of claim 1, wherein the time period is a predetermined time period.

3. The system of claim 1, wherein the controller is configured to signal the current generator to generate the set of currents in a closed loop mode of operation, an open loop mode of operation, or other mode of operation.

4. The system of claim 1, wherein the controller is configured to signal the current generator to cause neural suppression in the neural tissue after the time period and without causing sensory deficit.

5. The system of claim 1, wherein the set of currents is sufficient to modulate electrically excitable tissue.

6. The system of claim 1 or 5, wherein the at least one implantable working electrode comprises a high charge capacity material.

7. The system of claim 1, wherein the controller is further configured to signal the current generator to generate a bias current.

8. The system of claim 7, wherein the bias current operates in an anodic polarity.

9. The system of claim 7, wherein the bias current operates in a cathodic polarity.

10. The system of claim 7, wherein the current generator is configured to generate the bias current that biases the working electrode voltages cathodically.

11. The system of claim 7, wherein the current generator is configured to generate the bias current that biases the indifferent electrode voltages anodically.

12. The system of any of claims 1-11, comprising at least two working electrodes.

13. The system of claim 1, wherein the initial polarity of at least one working electrode is cathodic.

14. The system of claim 1, wherein the initial polarity of at least one working electrode is anodic.

15. The system of any of claims 1-14, wherein the indifferent electrode is a skin surface electrode.

16. The system of any of claims 1-15, wherein the indifferent electrode is a transcutaneous electrode or an implanted electrode.

17. The system of any of claims 1-16, wherein the indifferent electrode comprises titanium.

18. The system of any of claims 1-17, wherein the indifferent electrode has a working surface area of at least about 10 cm².

19. The system of any of claims 1-18, wherein the indifferent electrode has a working surface area of at least about 50 cm².

20. The system of any of claims 1-19, wherein the system is configured to generate a block of the excitable tissue.

21. The system of any of claims 1-20, wherein the at least one implantable working electrode comprises titanium nitride, tantalum, MP35N, platinum iridium, stainless steel, and/or a combination thereof.

22. The system of any of claims 1-21, wherein the at least one implantable working electrode comprises platinum-iridium coated with titanium nitride.

23. The system of any of claims 1-22, wherein the system does not include any blocking capacitors.

24. The system of any of claims 1-23, wherein the bias current is fixed.

25. The system of any of claims 1-24, wherein the bias current is variable.

26. The system of claim 25, wherein the bias current is variable over a cycle period or from a first cycle to a second cycle.

27. The system of claim 25 wherein the controller is configured to adjust the set of working electrode currents to modulate the bias current based on receiving data relating to one or any combination of capacitance, maximum current, and voltage.

28. The system of any of claims 1-27, wherein the bias current is between about −10 μA and about −1 mA.

29. The system of any of claims 1-28, wherein the bias current is between about −10 μA and about −100 μA.

30. The system of any of claims 1-29, wherein the bias current is evenly or unevenly split between a plurality of the implantable working electrodes.

31. The system of any of claims 1-30, further configured to inhibit water electrolysis in the patient.

32. The system of any of claims 1-31, further configured to inhibit one or more of: corrosion of the working electrode, an undesired chemical reaction, hydrogen embrittlement, creation of a reactive oxygen species, or creation of hydrogen peroxide.

33. The system of any of claims 1-32, wherein the current generator is configured to generate DC current.

34. The system of any of claims 1-33, wherein the current generator is configured to generate high frequency AC current.

35. The system of any of claims 1-34, wherein the working electrode is configured to deliver at least about 2,000 μC of charge into excitable tissue without damaging the excitable tissue.

36. The system of any of claims 1-35, wherein the system is devoid of any mechanically moving parts.

37. The system of any of the previous claims, wherein the time period is selected from the group consisting of: at least 5 minutes, at least 10 minutes, at least 20 minutes, at least 30 minutes, at least 45 minutes, at least one hour, at least two hours, at least 3 hours, at least four hours, and at least 5 hours.

38. The system of any of the previous claims, wherein the threshold current amplitude between 700 and 1000 μA.

39. The system of any of the previous claims, wherein neural suppression comprises pain suppression.

40. The system of any of the previous claims, wherein the sensory deficit is selected from the group consisting of: numbness, anesthesia, and paresthesia.

41. A method for electrically modulating tissue, comprising:
providing a current generator;
signaling the current generator to generate a set of currents with a set of initial polarities to be delivered to the working electrodes for a time period, wherein the magnitudes of the currents are less than a threshold current amplitude corresponding to a current amplitude required to achieve acute blockade in neural tissue; and
causing neural suppression in the neural tissue after the time period.

42. A system for electrically modulating tissue, comprising:
a current generator;
at least one implantable working electrode, the at least one implantable working electrode configured to be in electrical communication with the current generator;
at least one indifferent electrode; and
a controller configured to signal the current generator to:
generate a set of currents with a set of initial polarities to be delivered to the working electrodes for a time period;
cause a reduction in hypersensitive neuron sensitivity; and
maintain an acute pain response.

43. The system of claim 42, wherein the time period is a predetermined time period.

44. The system of claim 42, wherein the controller is configured to signal the current generator to generate the set of currents in a closed loop mode of operation, an open loop mode of operation, or other mode of operation.

45. The system of claim 42, wherein the set of currents is sufficient to modulate electrically excitable tissue.

46. The system of claim 42 or 45, wherein the at least one implantable working electrode comprises a high charge capacity material.

47. The system of claim 42 or 45, wherein the bias current operates in an anodic polarity.

48. The system of claim 42 or 45, wherein the bias current operates in a cathodic polarity.

49. The system of claim 42, wherein the set of currents are configured to generate the bias current that biases the working electrode voltages cathodically.

50. The system of claim 42, wherein the set of currents is configured to generate the bias current that biases the working electrode voltages anodically.

51. The system of claim 42, wherein the set of currents are configured to generate the bias current that biases the indifferent electrode voltages cathodically.

52. The system of claim 42, wherein the set of currents is configured to generate the bias current that biases the indifferent electrode voltages anodically.

53. The system of any of claims 42-52, comprising at least two working electrodes.

54. The system of claim 42, wherein the initial polarity of at least one working electrode is cathodic.

55. The system of claim 42, wherein the initial polarity of at least one working electrode is anodic.

56. The system of any of claims 42-55, wherein the indifferent electrode is a skin surface electrode.

57. The system of any of claims 42-56, wherein the indifferent electrode is a transcutaneous electrode or an implanted electrode.

58. The system of any of claims 42-57, wherein the indifferent electrode comprises titanium.

59. The system of any of claims 42-58, wherein the indifferent electrode has a working surface area of at least about 10 cm².

60. The system of any of claims 42-59, wherein the indifferent electrode has a working surface area of at least about 100 cm².

61. The system of any of claims 42-60, wherein the system is configured to generate a block of the excitable tissue.

62. The system of any of claims 42-61, wherein the high charge capacity material comprises titanium nitride, tantalum, MP35N, and/or a combination thereof.

63. The system of any of claims 42-62, wherein the high charge capacity material comprises tantalum coated with titanium nitride.

64. The system of any of claims 42-63, wherein the system does not include any blocking capacitors.

65. The system of any of claims 42-64, wherein the bias current is fixed.

66. The system of any of claims 42-65, wherein the bias current is variable.

67. The system of claim 65 wherein the controller is configured to adjust the set of working electrode currents to modulate the bias current based on receiving data relating to one or any combination of capacitance, maximum current, and voltage.

68. The system of any of claims 42-67, wherein the bias current is between about −10 μA and about −1 mA.

69. The system of any of claims 42-68, wherein the bias current is between about −10 μA and about −100 μA.

70. The system of any of claims 42-69, wherein the bias current is about −42 μA.

71. The system of any of claims 42-70, wherein the bias current is evenly or unevenly split between a plurality of the implantable working electrodes.

72. The system of any of claims 42-71, configured to inhibit water electrolysis in the patient.

73. The system of any of claims 42-72, configured to inhibit corrosion of the working electrode.

74. The system of any of claims 42-73, wherein the current generator is configured to generate DC current.

75. The system of any of claims 42-74, wherein the current generator is configured to generate high frequency AC current.

76. The system of any of claims 42-75, wherein the working electrode is configured to deliver at least about 2,000 μC of charge into excitable tissue without damaging the excitable tissue.

77. The system of any of claims 42-76, wherein the system is devoid of any mechanically moving parts.

78. The system of any of the previous claims, wherein the predetermined time period is selected from the group consisting of: at least 5 minutes, at least 10 minutes, at least 20 minutes, at least 30 minutes, at least 45 minutes, at least one hour, at least two hours, at least 3 hours, at least four hours, and at least 5 hours.

79. A method for electrically modulating tissue, comprising:
providing a current generator;
signaling the current generator to generate a set of currents with a set of initial polarities to be delivered to the working electrodes for a time period;
causing a reduction in hypersensitive neuron sensitivity; and
maintaining an acute pain response.

80. A system for electrically modulating tissue, comprising:
a current generator;
at least one implantable working electrode, the at least one implantable working electrode configured to be in electrical communication with the current generator;
at least one indifferent electrode; and
a controller configured to signal the current generator to:
generate a set of currents with a set of initial polarities and current amplitude levels associated with a partial neural conduction block of neural tissue to be delivered to the working electrodes;
cause a partial neural conduction block in the neural tissue in response to said set of currents.

81. The system of claim 80, wherein the set of currents is sufficient to modulate electrically excitable tissue.

82. The system of claim 80 or 81, wherein the at least one implantable working electrode comprises a high charge capacity material.

83. The system of claim 80 or 81, wherein the bias current operates in an anodic polarity.

84. The system of claim 80 or 81, wherein the bias current operates in a cathodic polarity.

85. The system of claim 80, wherein the set of currents are configured to generate the bias current that biases the working electrode voltages cathodically.

86. The system of claim 80, wherein the set of currents is configured to generate the bias current that biases the working electrode voltages anodically.

87. The system of claim 80, wherein the set of currents are configured to generate the bias current that biases the indifferent electrode voltages cathodically.

88. The system of claim 80, wherein the set of currents is configured to generate the bias current that biases the indifferent electrode voltages anodically.

89. The system of any of claims 80-88, comprising at least two working electrodes.

90. The system of claim 80, wherein the initial polarity of at least one working electrode is cathodic.

91. The system of claim 80, wherein the initial polarity of at least one working electrode is anodic.

92. The system of any of claims 80-91, wherein the indifferent electrode is a skin surface electrode.

93. The system of any of claims 80-92, wherein the indifferent electrode is a transcutaneous electrode or an implanted electrode.

94. The system of any of claims 80-93, wherein the indifferent electrode comprises titanium.

95. The system of any of claims 80-94, wherein the indifferent electrode has a working surface area of at least about 10 cm².

96. The system of any of claims 80-95, wherein the indifferent electrode has a working surface area of at least about 100 cm².

97. The system of any of claims 80-96, wherein the system is configured to generate a block of the excitable tissue.

98. The system of any of claims 80-97, wherein the high charge capacity material comprises titanium nitride, tantalum, MP35N, and/or a combination thereof.

99. The system of any of claims 80-98, wherein the high charge capacity material comprises tantalum coated with titanium nitride.

100. The system of any of claims 80-99, wherein the system does not include any blocking capacitors.

101. The system of any of claims 80-100, wherein the bias current is fixed.

102. The system of any of claims 80-101, wherein the bias current is variable.

103. The system of claim 101 wherein the controller is configured to adjust the set of working electrode currents to modulate the bias current based on receiving data relating to one or any combination of capacitance, maximum current, and voltage.

104. The system of any of claims 80-103, wherein the bias current is between about −10 μA and about −1 mA.

105. The system of any of claims 80-104, wherein the bias current is between about −10 μA and about −100 μA.

106. The system of any of claims 80-105, wherein the bias current is about −42 μA.

107. The system of any of claims 80-106, wherein the bias current is evenly or unevenly split between a plurality of the implantable working electrodes.

108. The system of any of claims 80-107, configured to inhibit water electrolysis in the patient.

109. The system of any of claims 80-108, configured to inhibit corrosion of the working electrode.

110. The system of any of claims 80-109, wherein the current generator is configured to generate DC current.

111. The system of any of claims 80-110, wherein the current generator is configured to generate high frequency AC current.

112. The system of any of claims 80-111, wherein the working electrode is configured to deliver at least about 2,000 μC of charge into excitable tissue without damaging the excitable tissue.

113. The system of any of claims 80-112, wherein the system is devoid of any mechanically moving parts.

114. The system of any of the previous claims, wherein the set of currents includes a positive plateau phase and a negative plateau phase, and wherein the current amplitude level is selected to cause a neural block during only the positive plateau phase.

115. The system of any of the previous claims, wherein the set of currents includes a positive plateau phase and a negative plateau phase, and wherein the current amplitude level is selected to cause a neural block during only the negative plateau phase.

116. The system of any of the previous claims, wherein the set of currents includes a positive plateau phase and a negative plateau phase, and wherein the current amplitude level is selected to cause a neural block during both the positive plateau phase and the negative plateau phase.

117. The system of any of the previous claims, wherein the set of currents is chosen to cause a neural block during both the positive plateau phase and the negative plateau phase, while allowing neural conduction during the transition period between phases.

118. The system of any of the previous claims, wherein the current amplitude level is linearly related to a percentage of partial block.

119. The system of any of the previous claims, wherein the current amplitude level is exponentially related to a percentage of partial block.

120. The system of any of the previous claims, wherein the controller is further configured to determine an indication of a level of neural blocking and adjust the amplitudes of the set of currents in response to the indication.

121. The system of any of the previous claims, wherein the controller is further configured to cause the partial neural conduction block to be eliminated within 10, 20, 30, 40, 50, 60, 70, 80, or 90 ms of stopping generation of the set of currents.

122. The system of any of the previous claims, wherein the controller is further configured to cause the partial neural conduction block to be eliminated within a period of time that is 50%, 60%, 70%, 80%, 90%, 100%, 110%, 120%, 130%, 140%, 150%, or 200% of a period of time that the partial neural conduction block is applied.

123. A method for electrically modulating tissue, comprising:
providing a current generator,
signaling the current generator to generate a set of currents with a set of initial polarities and current amplitude levels associated with a partial neural conduction block of neural tissue to be delivered to the working electrodes; and
causing a partial neural conduction block in the neural tissue in response to said set of currents.

124. A system for electrically modulating tissue, comprising any one or more of the embodiments described in the disclosure.

125. A method for electrically modulating tissue, comprising any one or more of the embodiments described in the disclosure.
